(19) United States
(12) Patent Application Publication
(10) Pub. No.: US 2025/0082501 A1
(43) Pub. Date: Mar. 13, 2025

(54) IMPLANTABLE SHUNTS WITH MULTI-LAYERED FLUID RESISTORS, AND ASSOCIATED SYSTEMS AND METHODS

(71) Applicant: Shifamed Holdings, LLC, Campbell, CA (US)

(72) Inventors: Tessa Bronez, Campbell, CA (US); Eric Schultz, Los Altos, CA (US); Robert Chang, Belmont, CA (US); Katherine Sapozhnikov, Campbell, CA (US); Tom Saul, Portland, OR (US); Michael Drews, Palo Alto, CA (US); Richard Lilly, San Jose, CA (US)

(21) Appl. No.: 18/290,595

(22) PCT Filed: Jul. 21, 2022
(86) PCT No.: PCT/US2022/037917

§ 371 Date: Jan. 19, 2024

Related U.S. Application Data
(60) Provisional application No. 63/224,075, filed on Jul. 21, 2021.

(51) Int. Cl. A61F 9/007

(52) U.S. Cl. CPC A61F 9/00781

(57) ABSTRACT

The present technology provides microfluidic shunting systems having multiple channels and associated methods of making the same. In some embodiments. the microfluidic shunting systems comprise a plurality of layers or drainage elements that are stacked and adhered together. In some embodiments, individual channels can be positioned in separate layers or drainage elements. and/or can span multiple layers or drainage elements.

### CROSS-REFERENCE TO RELATED APPLICATION(S)

The present application claims priority to U.S. Provisional Patent Application No. 63/224,075, filed Jul. 21, 2021, the disclosure of which is incorporated by reference herein in its entirety.

### TECHNICAL FIELD

The present technology generally relates to implantable medical devices and, in particular, to multi-layered fluid resistors for controlling fluid flow between a first body region and a second body region of a patient.

### BACKGROUND

Implantable shunting systems are widely used to treat a variety of patient conditions by shunting fluid from a first body region/cavity to a second body region/cavity. For example, shunting systems have been proposed for treating glaucoma. The flow of fluid through the shunting systems is primarily controlled by the pressure gradient across the shunt and the physical characteristics of the flow path defined through the shunt (e.g., the resistance of the shunt lumen). Conventional, early shunting systems (sometimes referred to as minimally invasive glaucoma surgery devices or “MIGS” devices) have shown clinical benefit; however, there is a need for improved shunting systems and techniques for addressing elevated intraocular pressure and risks associated with glaucoma. For example, there is a need for shunting systems capable of adjusting the therapy provided, including the flow rate between the two fluidly connected bodies. As another example, there is a need for a shunting system capable of being modified after manufacture (e.g., in the clinic) to personalize the system for the patient and/or as part of the clinician's plan for the implant procedure.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1 is a perspective view of a drainage assembly configured in accordance with select embodiments of the present technology.

FIG. 2A is a perspective view of a drainage assembly configured in accordance with select embodiments of the present technology.

FIG. 2B is a partially exploded view of the drainage assembly of FIG. 2A.

FIG. 2C is an exploded view of the drainage assembly of FIG. 2A.

FIG. 2D is a perspective view of a drainage element of the drainage assembly of FIG. 2A, with other aspects of the drainage assembly omitted for the purpose of clarity.

FIGS. 3A and 3B illustrate drainage elements configured in accordance with select embodiments of the present technology.

FIG. 4A illustrates a drainage element including one or more alignment apertures and configured in accordance with select embodiments of the present technology.

FIG. 4B illustrates a plurality of the drainage elements of FIG. 4A.

FIGS. 5A and 5B illustrate an actuation assembly configured in accordance with select embodiments of the present technology.

FIGS. 6A and 6B are top views of a flow control system including the drainage assembly of FIG. 1 and the actuation assembly of FIGS. 5A and 5B and configured in accordance with select embodiments of the present technology.

FIGS. 7A and 7B illustrate an actuator for controlling the flow of fluid in a shunting system and configured in accordance with select embodiments of the present technology.

FIG. 8A illustrates a shunting system configured in accordance with select embodiments of the present technology.

FIG. 8B illustrates a fluid resistor network of the shunting system shown in FIG. 8A and configured in accordance with select embodiments of the present technology.

FIG. 8C is a schematic diagram of the fluid resistor network shown in FIG. 8B.

FIG. 8D is an exploded isometric view of the shunting system shown in FIG. 8A.

FIG. 9A illustrates another shunting system configured in accordance with select embodiments of the present technology.

FIG. 9B is a schematic diagram of a fluid resistor network of the shunting system shown in FIG. 9A.

FIG. 9C is an exploded isometric view of the shunting system shown in FIG. 9A.

FIG. 10A illustrates yet another shunting system configured in accordance with select embodiments of the present technology.

FIG. 10B is a schematic diagram of a fluid resistor network of the shunting system shown in FIG. 10A.

FIG. 11 is a cross-sectional view of a portion of a fluid resistor network configured in accordance with embodiments of the present technology.

### DETAILED DESCRIPTION

Intraocular shunts for treating glaucoma are implanted in a patient's eye to drain aqueous from a patient's anterior chamber, thereby reducing intraocular pressure. Such shunts are necessarily small, both because of the target implant site and because smaller intraocular shunts tend to be associated with fewer side effects, including decreased interference with patient vision, less tissue irritation, and the like. Indeed, the current commercialized class of intraocular shunts known as minimally invasive glaucoma surgery (“MIGS”) devices have recognized that it is beneficial for shunts to be as small as possible while still providing an effective therapy. As a result, most MIGS devices have cross-sectional dimensions (e.g., diameter, width, height, etc.) of less than about 1 mm or smaller.

In addition to being small, it is also beneficial for shunts to provide more than one flow pathway. Having multiple flow pathways through a single shunt is advantageous because flow patency is maintained if one or more of the flow pathways become blocked by cellular debris. Having multiple flow pathways through a single shunt is also advantageous because, in certain embodiments, flow through the shunt can be selectively diverted from or between specific channels to provide a titratable fluid resistance, and thus a titratable therapy. Of course, adding more channels generally increases the size of the overall shunt. As a result, there is a conflict between minimizing the size of the shunt and providing multiple channels through the shunt to optimize therapy. Thus, a need exists to provide shunting systems that can accommodate multiple flow pathways while retaining a relatively small footprint.

The present technology provides microfluidic shunting systems having multiple channels and associated methods of making such systems. In some embodiments, the shunting systems can be composed of a plurality of layers or elements that are sealingly coupled together. As described in detail throughout this Detailed Description, individual channels can be positioned in separate layers or elements and/or can span multiple layers or elements. As described below, this is expected to increase the number of channels that can fit within the shunting system, while keeping the size of the overall system relatively small.

In some embodiments, the microfluidic shunting systems are generally flat, such that they have a width to height ratio of between 5:1 and 50:1. As a result, the microfluidic shunting systems can be composed of a plurality of discrete flat layers stacked and adhered together, with each individual layer having a height of, e.g., less than about 500 μm. As described in detail below, the shunts can include a plurality of channels. Advantageously, in some embodiments individual channels are positioned in separate layers, and/or individual channels span multiple layers, such that the channels can be vertically stacked. As will be apparent from the description below, this is expected to increase the number of channels that can fit within the shunting system, while keeping the overall system relatively small.

In some embodiments, the adjustable shunting systems include drainage assemblies having one or more drainage elements or cartridges that can be “stacked” or otherwise aligned in a modular fashion. Each of the drainage elements can include a drainage inlet, a drainage lumen portion, and a channel that fluidly couples the drainage inlet to the drainage lumen portion. Fluid can enter the drainage element via the fluid inlet and travel through the channel to the drainage lumen portion. The drainage elements can be stacked or otherwise oriented such that a plurality of drainage lumen portions of individual drainage elements align to form a common drainage lumen. The fluid entering the drainage elements via the fluid inlets can therefore be collected in the common drainage lumen, which can direct the collected fluid to an exit in a desired outflow region.

In some embodiments, each of the channels extending between a fluid inlet and a drainage lumen portion can have a length that imparts a particular fluid resistance. In at least some embodiments, for example, a first drainage element can have a first channel having a first length corresponding to a first fluid resistance, and a second drainage element can have a second channel having a second length greater than the first length, the second length corresponding to a second fluid resistance greater than the first fluid resistance. Thus, at a given pressure, the first drainage element can be associated with a first fluid flow rate, and the second drainage element can be associated with a second fluid flow rate less than the first fluid flow rate. Accordingly, an adjustable shunting system including the first and second drainage elements can be used to provide a titratable therapy, e.g., based on the varied flow rates of the drainage elements.

In some embodiments, the present technology can further include an actuation assembly for controlling the flow of fluid through the drainage assemblies. The actuation assembly can include, for example, one or more actuators configured to control the flow of fluid through the drainage assembly. In particular, each actuator can include a control element corresponding to and configured to interface with one of the drainage elements. For example, each control element can be configured to interface with a corresponding drainage inlet of the drainage element(s). The actuator can also have a first actuation element and a second actuation element configured to move the control element between (i) a first position in which the control element does not substantially prevent or hinder fluid flow through the corresponding fluid inlet (e.g., the drainage inlet is accessible) and (ii) a second position in which the control element substantially prevents or hinders fluid flow through the corresponding drainage inlet (e.g., the control element covers or blocks the drainage inlet). Accordingly, the actuation assembly can be used to control access to the drainage assembly, e.g., to provide a titratable therapy.

As described in greater detail below, it is expected that drainage assemblies having one or more drainage elements or cartridges configured in accordance with the present technology may exhibit one or more advantageous characteristics that improve operation of adjustable shunting systems. For example, the drainage assemblies described herein can be relatively compact while still having channels of sufficient length to impart a desired fluid resistance. Additionally, at least some of the drainage assemblies and/or drainage elements are expected to be easier and/or less costly to manufacture compared to traditional drainage assemblies and drainage elements. In some embodiments, for example, the drainage elements can include one or more alignment features or apertures configured to stack or otherwise align one or more first elements (e.g., a first drainage inlet, a first drainage lumen portion, etc.) of a first drainage element with one or more second elements (e.g., a second drainage inlet, a second drainage lumen portion, etc.) of a second drainage element. Of course, the present technology may also provide additional advantageous characteristics not expressly described herein.

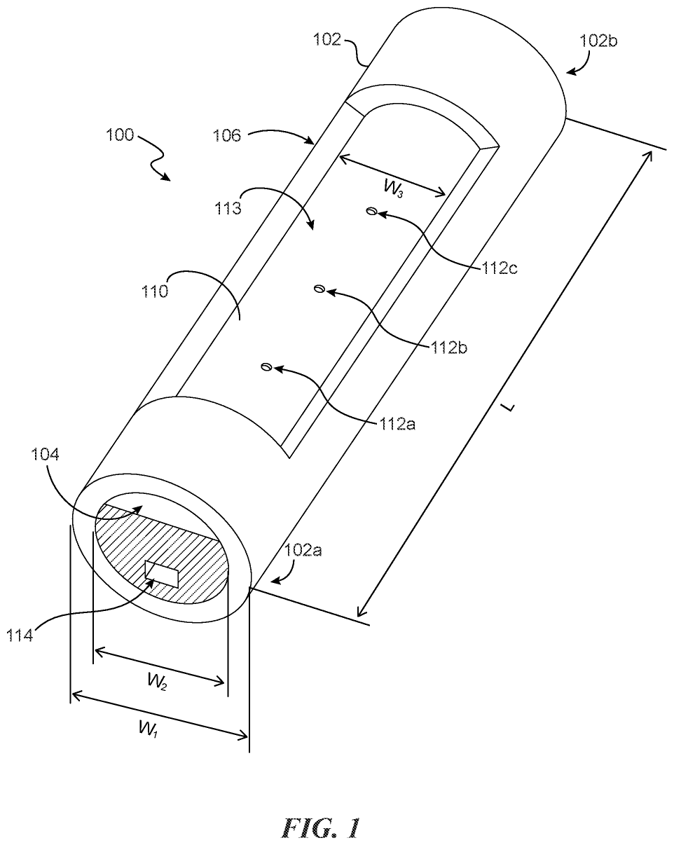

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1A-11.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment Furthermore, the particular features or characteristics may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

Although certain embodiments herein are described in terms of shunting fluid from an anterior chamber of an eye, one of skill in the art will appreciate that the present technology can be readily adapted to shunt fluid from and/or between other portions of the eye (including the posterior chamber), or, more generally, from and/or between a first body region and a second body region. Moreover, while the certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

The headings below are provided by way of convenience only and are not to be used to interpret the scope of the claimed technology.

### A. Shunting Systems with Stackable Channels and Multi-Layered Resistors

FIG. 1 is a perspective view of a drainage or shunting assembly 100 (“the assembly 100”) configured in accordance with select embodiments of the present technology. The assembly 100 can include an elongate carriage or housing element 102 having a first end portion 102a and a second end portion 102b opposite the first end portion 102a. The carriage element 102 can be at least partially hollow, such that the carriage element 102 can include an interior 104 extending at least partially between the first end portion 102a and the second end portion 102b. In some embodiments, the carriage element 102 can include a slot or gap 106 extending longitudinally and/or radially through the carriage element 102 and at least partially between the first and second end portions 102a-b. The slot 106 can at least partially expose the interior 104 of the carriage element 102, e.g., such that one or more objects positioned within the interior 104 can contact, be coupled to (e.g., fluidly coupled to) or otherwise interface with an environment external to the carriage element 102 through the slot 106.

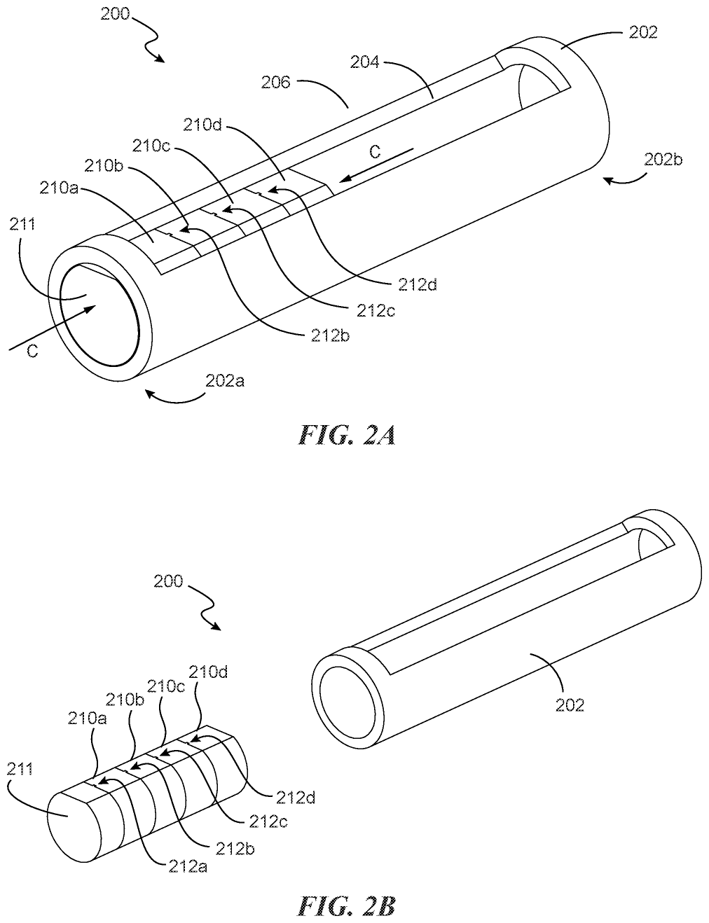

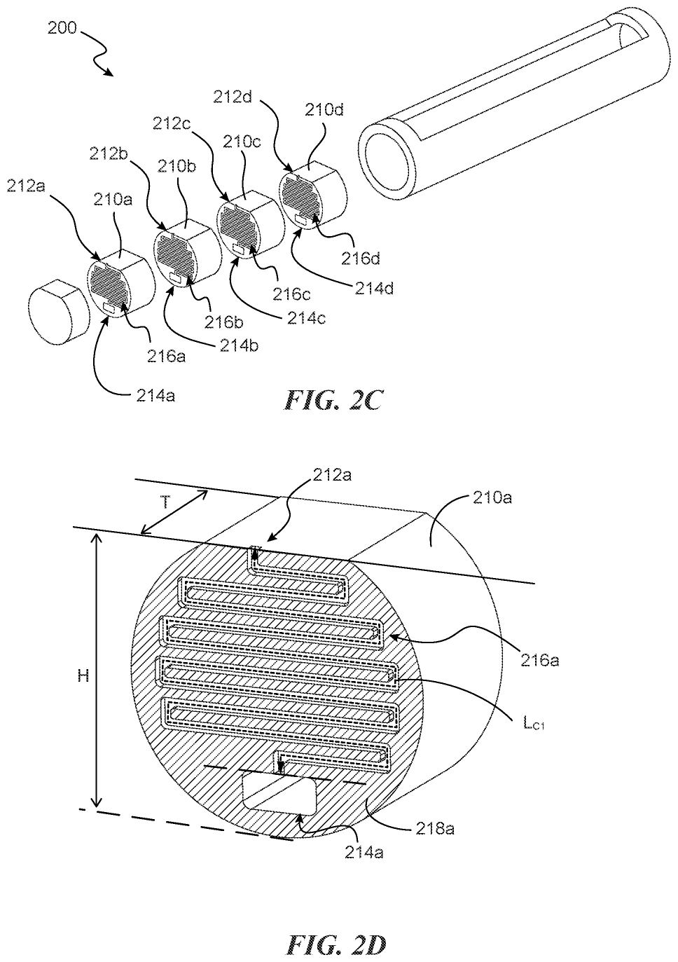

The assembly 100 can further include one or more drainage elements 110. The drainage element(s) 110 can be positioned at least partially within the interior 104 of the carriage element 102, e.g., at least partially between the first and second end portions 102a-b, such that the carriage element 102 can carry, transport, and/or otherwise house the drainage element(s) 110. The drainage element(s) 110 can further include one or more fluid or drainage apertures or inlets 112. In the illustrated embodiment, for example, the drainage element(s) 110 include a first drainage inlet 112a, a second drainage inlet 112b, and a third drainage inlet 112c. The drainage inlets 112a-112c can be fluidly connected to a primary drainage lumen or collection chamber 114 (“the lumen 114”) by one or more fluid channels (not shown in FIG. 1) extending through the drainage element(s) 110, as shown in and described with reference to FIGS. 2C and 2D. Accordingly, fluid flowing into the assembly 100 via the drainage inlets 112a-112c drains to the lumen 114 via the channels (FIGS. 2C and 2D). In some embodiments, one of the end portions 102a, b can be blocked or sealed to reduce or prevent fluid in the lumen 114 from exiting the assembly 100 via the blocked end portion 102a,b. In the illustrated embodiment, for example, the second end portion 102b is blocked such that, once fluid enters the lumen 114, the fluid flows toward the first end portion 102a of the carriage element 102, e.g., to exit the assembly 100.

In some embodiments, the assembly 100 can be coupled to an outflow tube or other fluid transporting element (not shown) for transporting fluid received within the lumen 114 via the fluid inlets 112a-112c to and/or toward a desired outflow location. For example, a proximal end portion of the outflow tube can be fluidly coupled to the lumen 114 at the first end portion 102a of the carriage element 102 such that the outflow tube receives fluid from the lumen 114, and a distal end portion of the outflow tube can be positioned at the desired outflow location. In other embodiments, the first end portion 102a of the assembly 100 can be configured to reside at a desired outflow location such that an outlet of the lumen 114 at the first end portion 102a of the carriage element 102 resides at the desired outflow location (e.g., omitting the need for an outflow tube). Accordingly, fluid in a first body region can enter the assembly 100 and pass through the fluid inlet(s) 112 to a second body region via the lumen 114 (and/or an outflow tube).

In some embodiments, the drainage element(s) 110 can form a fluid seal with the carriage element 102. In the illustrated embodiment, for example, an outer surface of the drainage element(s) 110 can form a fluid seal with an inner surface of the carriage element 102, e.g., to reduce or prevent fluid from entering a space between the drainage element(s) 110 and the carriage element 102. This can reduce or prevent leaks or other uncontrolled fluid flow throughout the assembly 100, such that fluid can travel through the assembly 100 only via one or more predetermined flow paths (e.g., through the drainage inlet(s) 112 and the corresponding channels (FIGS. 2C-2D) and lumen(s) 114).

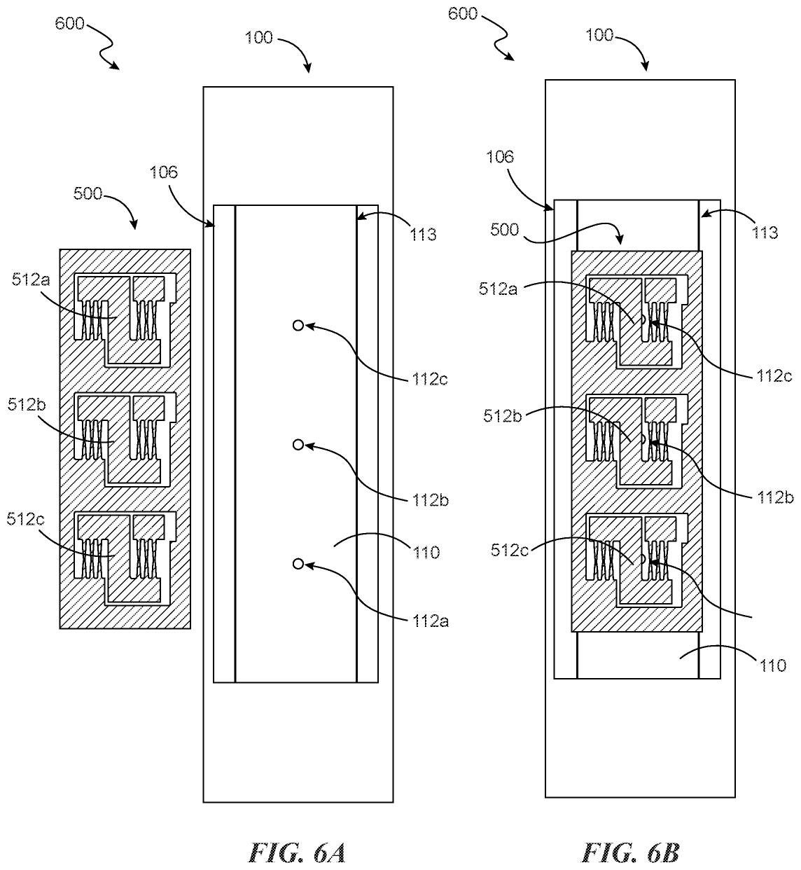

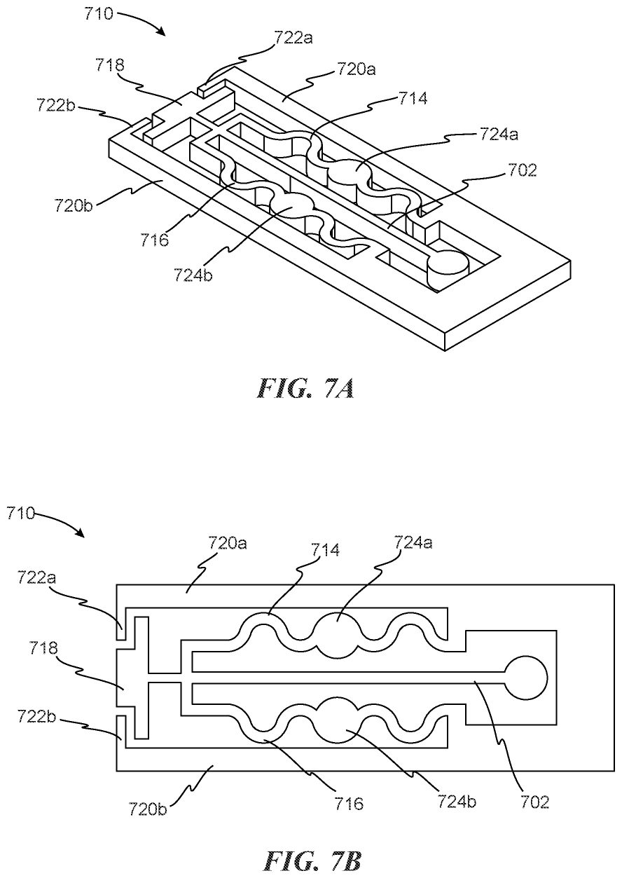

In some embodiments, the drainage element(s) 110 can include an interface side or surface 113 that is generally or substantially flat or planar. The interface surface 113 can correspond (e.g., be sized, positioned, and/or aligned) to the slot 106, such that the interface surface 113 and the slot 106 can have a generally similar or the same width W₃. In some embodiments, the interface surface 113 can have a first width, and the interior of the slot 106 can have a second width less than or greater than the first width. Additionally, the drainage inlet(s) 112 can be positioned on the interface surface 113 and/or oriented towards the slot 106. Accordingly, the interface surface 113 can be used to align and/or position individual ones of the drainage element(s) 110 and/or the drainage inlet(s) 112 relative to each other and/or the slot 106. As described in greater detail below and with reference to FIGS. 6A-7B, an actuation assembly (not shown in FIG. 1) can be coupled to and/or interface with the interface surface 113 and configured to control the flow of fluid through the drainage inlet(s) 112.

Although the carriage element 102 is depicted as having a circular shape in FIG. 1, in other embodiments the carriage element 102 can have an oval shape, a triangular shape, a square shape, a rectangular shape, a pentagonal shape, a hexagonal shape, a rectilinear shape, or any other suitable shape. The drainage element(s) 110 can be shaped to at least partially correspond to the shape of the carriage element 102. In the illustrated embodiment, for example, the carriage element 102 has a circular shape, and the drainage element(s) 110 have an at least partially circular shape. In other embodiments, however, the drainage element(s) 112 can have an at least partially oval shape, an at least partially triangular shape, an at least partially square shape, an at least partially rectangular shape, an at least partially pentagonal shape, an at least partially hexagonal shape, an at least partially rectilinear shape, or any other suitable shape. Additionally, although the drainage element(s) 110 are depicted as including three fluid inlets 112a-c in FIG. 1, in other embodiments the drainage element(s) 110 can include more or fewer drainage inlets 112. In at least some embodiments, for example, the drainage element(s) 110 can include one, two, three, four, five, six, seven, eight, nine, ten, or any other suitable number of drainage inlets 112.

The carriage element 102 can be formed from polymer materials, such as polyimide or polyether ether ketone (PEEK), metals, such as stainless steel, nitinol, or titanium, a combination thereof (e.g., nitinol), and/or any other suitable material. The drainage element(s) 110 can be formed from polymers (e.g., PEEK or polyimide), glass, metals, elastomers (e.g., silicone elastomers, thermoplastic elastomers (TPE), or urethane), a combination thereof, and/or any other suitable material.

In some embodiments, the carriage element 102 can have a length L between about 1 mm and about 20 mm, such as at least 1 mm, at least 2 mm, at least 3 mm, at least 4 mm, at least 5 mm, at least 6 mm, at least 7 mm, at least 8 mm, at least 9 mm, at least 10 mm, or any other suitable length. The carriage element 102 can have an outer width (e.g., a diameter) W₁ between about 450 μm and about 1.5 mm, such as at least 500 μm, at least 550 um, at least 600 μm, at least 650 μm, at least 700 μm, at least 750 μm, at least 1 mm, and/or any other suitable outer width W₁. The carriage element 102 (e.g., the interior 104) can have an inner width W₂ between about 250 μm and about 650 μm, such as up to 300 μm, up to 350 μm, up to 400 μm, up to 450 μm, up to 500 μm, up to 550 μm, up to 600 μm, and/or any other suitable inner width W₂. The drainage element(s) 110 can have an outer width generally similar to or the same as the inner width W₂, such that the drainage element(s) 110 can be positioned within the carriage element 102 as described above. In at least some embodiments, for example, the drainage element(s) 110 can have an outer width that is between 100% and about 80% of the inner width W₂, such as at least 99%, 98%, 97%, 96%, 95%, 90%, and/or any other suitable percent of the inner width W₂.

FIG. 2A is a perspective view of a drainage assembly 200 (“the assembly 200”) configured in accordance with embodiments of the present technology. FIG. 2B is a partial exploded view of the assembly 200 of FIG. 2A, FIG. 2C is an exploded view of the assembly 200 of FIG. 2A, and FIG. 2D is a perspective view of a drainage element 210 with other aspects of the assembly 200 omitted for the purpose of clarity. The assembly 200 and/or the components thereof can be generally similar to or the same as the assembly 100 of FIG. 1. Accordingly, like numbers are used to indicate like components (e.g., drainage element(s) 110 versus drainage element(s) 210), and a discussion of the assembly 200 will be limited to those features that differ from the assembly 100 of FIG. 1, and other aspects necessary for context.

Referring first to FIGS. 2A and 2B together, the assembly 200 can include a plurality of drainage elements 210 (which can also be referred to as cartridges, modular elements, stackable elements, layers, or the like). In the illustrated embodiment, for example, the assembly 200 includes a first drainage element 210a, a second drainage element 210b, a third drainage element 210c, and a fourth drainage element 210d. As best seen in FIG. 2B, the drainage elements 210a-d can be stacked, aligned, or layered (e.g., linearly, axially, in sequence, etc.) relative to each other. As shown in FIG. 2A, the drainage elements 210a-d can be positioned within a cavity or interior 204 defined by a carriage element 202 having a slot 206. Although the drainage element(s) 210 in the illustrated embodiment are shown as only occupying a portion of the interior 204 of the carriage element 202, in some embodiments the drainage element(s) 210 occupy the entire or substantially the entire portion of the interior 204 having the slot 206 (e.g, to prevent fluid from flowing into the interior 204 without passing through the drainage elements 210). For example, the interior 204 can be filled (e.g., completely filled) or otherwise occupied by the drainage elements 210, e.g., by adding additional drainage elements 210, increasing a thickness of the drainage elements 210, and/or reducing the length L (FIG. 1) of the carriage element 202. In other embodiments, a plug or other solid feature (not shown) without any fluid inlets can be added to the unoccupied space shown in FIG. 2A to prevent fluid from flowing into the interior 204 without passing through the drainage elements.

In some embodiments, the drainage elements 210 can be fluidically sealed to one another when in a stacked configuration as shown in FIGS. 2A and 2B. Without being bound by theory, this is expected to prevent or at least reduce fluid from leaking between adjacent drainage elements 210. In the illustrated embodiment, for example, a first fluid seal can be formed between the first and second drainage elements 210a-b, a second fluid seal can be formed between the second and third drainage elements 210b-c, and a third fluid seal can be formed between the third and fourth drainage elements 210c-d. In some embodiments, individual drainage element(s) 210 can be pressed (e.g., compressed in a longitudinal direction indicated by arrows C in FIG. 2A) or otherwise held together by the carriage element 202 to form the fluid seal(s). For example, a friction force between the drainage element(s) 210 and the carriage element 202 can maintain the positions of the drainage elements 210 relative to each other, e.g, to form the fluid seal(s) between individual ones of the drainage elements(s) 210. In some embodiments, the fluid seal can be formed via adhesives, ultrasonic welding, thermal fusion, thermal reflow, and/or any other suitable process or technique. In some embodiments, one or more of the drainage elements 210 can be self-adhering, such that the self-adhering drainage element can form (e.g., automatically form) the fluid seal when in contact with one or more of the other drainage elements. For example, one or more of the drainage elements 210 may be composed of a self-bonding material that forms a bond or seal with adjacent drainage elements under certain conditions (e.g., in a vacuum).

Each of the drainage elements 210a-d can include one or more drainage inlets 212. In the illustrated embodiment, for example, the first drainage element 210a includes a first drainage inlet 212a (best seen in FIG. 2B), the second drainage element 210b includes a second drainage inlet 212b, the third drainage element 210c includes a third drainage inlet 212c, and the fourth drainage element 210d includes a fourth drainage inlet 212d. In some embodiments, the assembly 200 further includes an endcap or retaining element 211. The endcap 211 can be positioned within the first end portion 202a and/or the second end portion 202b of the carriage element 202. The endcap 211 can be generally similar to or the same as the drainage element(s) 210, but can lack (e.g., not include) a fluid inlet and/or a drainage lumen portion. In some embodiments, the endcap 211 can be configured to retain the drainage element(s) 210 in (e.g., within the interior 204 of) the carriage element 202. Accordingly, the endcap 211 can reduce or prevent movement of the drainage element(s) 210 within the interior 204, can maintain the drainage element(s) 210 in a compressed state, and/or can maintain the fluid seal(s) between individual ones of the drainage element(s) 210. In some embodiment, the endcap 211 can form a fluid seal with the drainage element(s) 210, as described previously. In the illustrated embodiment, for example, the endcap 211 can form a fifth fluid seal with the first drainage element 210a. In some embodiments, the endcap 211 can at least partially cover or block the drainage lumen portion(s) 214 (FIGS. 2C, and 2D), such that the endcap 211 can reduce or prevent fluid flow through the first end portion 202a and/or the second end portion 202b. In the illustrated embodiment, for example, the endcap 221 can block or seal the first end portion 202a such that, once fluid enter the lumen portion(s) 214 (FIGS. 2C and 2D), the fluid cannot flow out of the drainage assembly 200 via the first end portion 202a. Rather, in the illustrated embodiment, the fluid flows toward the second end portion 202b of the carriage element 202 to exit the drainage assembly (e.g., via an outflow tube; not shown).

Referring next to FIG. 2C, the drainage element(s) 210 can include one or more drainage lumen or fluid connection portions. In the illustrated embodiment, for example, the first drainage element 210a includes a first drainage lumen portion 214a, the second drainage element 210b includes a second drainage lumen portion 214b, the third drainage element 210c includes a third drainage lumen portion 214c, and the fourth drainage element 210d includes a fourth drainage lumen portion 214d (“the drainage lumen portion(s) 214”). When the drainage element(s) 210 are positioned within the carriage element 202 (e.g., as shown in FIG. 2A), each of the drainage lumen portion(s) 214 can be generally or substantially aligned, e.g., such that the drainage lumen portions 214a-d align to collectively form a single or common drainage lumen (e.g., the drainage lumen 114 of FIG. 1). Accordingly, the plurality of drainage lumen portions 214 can collectively form a drainage lumen for transporting fluid received from the fluid inlets 212 through the drainage assembly 200.

Each drainage element 210 further includes a flow path or channel 216 fluidly coupling the drainage inlet 212 to the corresponding drainage lumen portion 214. In the illustrated embodiment, for example, the first drainage element 210a includes a first channel 216a, the second drainage element 210b includes a second channel 216, the third drainage element 210c includes a third channel 216c, and the fourth drainage element includes a fourth channel 216d (“the channel(s) 216”). Accordingly, the first channel 216a can fluidly couple the first drainage inlet 212a to the first drainage lumen portion 214a, the second channel 216b can fluidly couple the second drainage inlet 212b to the second drainage lumen portion 214b, etc. As described below, the channels 216 provide a fluid resistance, and therefore can be referred to herein as a “fluid resistor network,” a “network of fluid resistors,” “parallel resistors,” or the like.

In operation, the carriage element 202 can be positioned such that it resides within a first body region or cavity (e.g., an anterior chamber). Accordingly, fluid in the first body region can enter the assembly 200 via one of the drainage inlets 212, pass through the corresponding channel 216 to the corresponding drainage lumen portion 214. The fluid then flows through the drainage lumen formed by the plurality of drainage lumen portions and into a second (e.g., outflow) body region (e.g., a sub conjunctival bleb space), and/or into an outflow tube, which can transport the fluid to the second body region as previously described. As described in greater detail below and with reference to FIG. 2D, each of the channels 216 can have a particular flow resistance such that the assembly 200 can provide a titratable therapy to a patient.

Although FIGS. 2A-2C depict the assembly 200 as having four drainage element(s) 210, in other embodiments the assembly 200 can include more or fewer drainage elements 210. In at least some embodiments, for example, the assembly 200 can include one, two, three, five, six, seven, eight, nine, ten, or more drainage elements 210.

FIG. 2D is an enlarged perspective view of the first drainage element 210a of FIG. 2C. Although described in the context of the first drainage element 210a, the following description of FIG. 2D applies equally to the second, third, and fourth drainage elements 210b-d of FIGS. 2A-2D. As shown, the first channel 216a can have a serpentine or rectilinear shape with a length L_{C1}, as measured by a distance fluid travels through the first channel 216a between the first drainage inlet 212a and the first drainage lumen portion 214a. Of note, the length L_{C1} can be greater than a height H of the first drainage element 210a, e.g., by virtue of the serpentine or rectilinear shape of the first channel 216a. In some embodiments, the length L_{C1} is selected/designed to provide a particular fluid resistance. For example, the length L_{C1} of the first channel 216a can be proportional to the resistance (e.g., a fluid or flow resistance) of the first channel 216a, such that the resistance of the first channel 216a increases as the length L_{C1} increases, and the resistance decreases as the length L_{C1} decreases (e.g., assuming the cross-sectional area of the channel 216a remains constant).

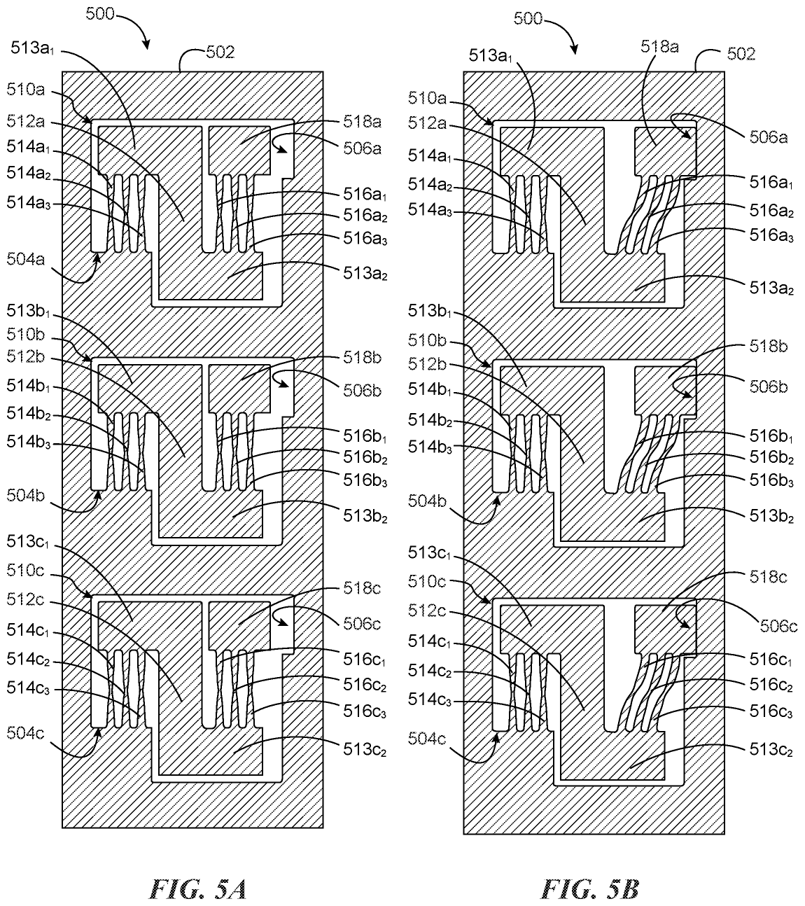

Each drainage element 210 can have channels 216 of varying lengths, and accordingly can have channels 216 having varying fluid resistances. For example, the first channel 216a can have a first length L_{C1} corresponding to a first resistance, and the second channel 216b can have a second length L_{C2} less than the first length L_{C1} and corresponding to a second resistance less than the first resistance. In such embodiments and under a given pressure, the assembly 200 can provide a first flow rate when fluid travels solely (or at least primarily) through the first channel 216a, and a second flow rate greater than the first flow rate when fluid travels solely (or at least primarily) through the second channels 216b. The third channel 216c and the fourth channel 216d can also have different lengths and thus different resistances to provide additional therapy options. Accordingly, the relative level of therapy provided by each drainage element 210 can be different, and the level of therapy provided by the assembly 200 can be adjusted by selectively permitting and/or blocking flow through various channels 216 and/or combinations of channels 216 (e.g., by selectively interfering with or permitting flow through individual drainage inlets 212, as described below with respect to FIGS. 5A-7B). In other embodiments, the channels 216 can have the same or generally similar geometric configurations such that they have the same or generally similar fluid resistances, and thus provide similar flow rates (for a given pressure).

In some embodiments, the level of fluid flow through the drainage element 210a is primarily controlled by the resistance of the channel 216a. For example, the drainage inlet 212a and the drainage lumen portion 214a can be sized (e.g., have a length, width, diameter, circumference, etc.) such that the drainage inlet 212 and the drainage lumen portion 214a do not provide substantial resistance to fluid flow therethrough relative to the resistance provided by the channel 216a. Accordingly, in at least some embodiments the length L_{C1} of the channel 216a primarily determines the fluid resistance of the drainage element 210a.

As best seen in FIG. 2D, the first drainage element 210a can have a thickness T that provides sufficient stability to the drainage element to prevent or at least partially reduce the deformability of the first drainage element 210a. For example, the thickness T can be between about 50 μm and about 500 μm, such as at least 50 μm, at least 100 μm, at least 150 μm, at least 200 μm, at least 250 μm, at least 300 μm, at least 350 μm, at least 400 μm, and/or any other suitable thickness. The thickness T can be based at least in part on the material forming the first drainage element 210a. For example, the first drainage element 210a can have a first thickness T₁ when the material is generally soft or compressible (e.g., a Young's modulus between about 0 gigapascals (GPa) and about 40 GPa, such as a Young's modulus of less than 40 GPa), and a second thickness T₂ less than the first thickness TI when the material is generally hard or incompressible (e.g., a Youngs modulus between about 100 GPa and about 500 GPA, such as a Young's modulus of greater than 100 GPa). In at least some embodiments, the length L (FIG. 1) of the assemblies 100, 200 can be generally similar to or the same as a combined or total length of the drainage element(s) 210 and the one or more end cap(s) 111, as measured by a sum of the thicknesses T of each of the drainage elements 210 and a thickness of the one or more end cap(s) 111.

In the embodiment illustrated by FIG. 2D, the first channel 216a is formed in a first surface 218a of the first drainage element 210a. Forming the channels 216 in the surfaces 218 of the drainage elements 210 can allow the channel 216 to be sealed (e.g., fluidly sealed) by the fluid seals formed between the individual drainage elements 210, as described previously and with reference to FIGS. 2A-2B. In some embodiments, the channels 216 can be formed in an interior of the drainage elements 210, such that the embodiment illustrated in FIG. 2D represents a half or cross-section of a drainage element 210. In at least some embodiments, the channel(s) 216 can be formed via laser cutting, machining, or any other suitable process or technique to remove material from the surface(s) 218 of the drainage element(s) 210. In at least some embodiments, the channel(s) 210 can be formed using a mold that corresponds to a desired length and/or shape of the channel(s) 210, e.g., as part of an additive manufacturing process.

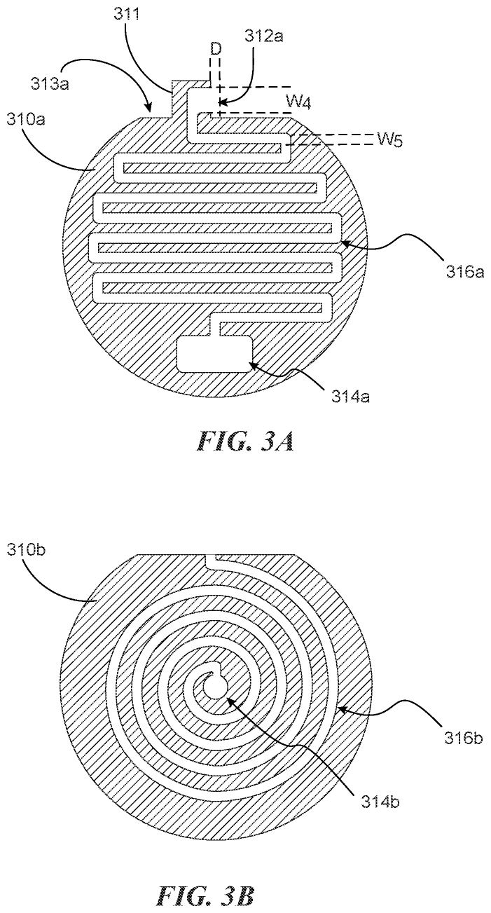

FIGS. 3A and 3B illustrate additional drainage elements 310a-b configured in accordance with select embodiments of the present technology. The drainage elements 310a-b and/or one or more components thereof can be generally similar to or the same as the drainage element(s) 110 of FIG. 1 and/or the drainage element(s) 210 of FIGS. 2A-2D. Accordingly, like numbers are used to reference like components (e.g., fluid inlet(s) 312 versus the fluid inlet(s) 112 of FIG. 1, the fluid inlet(s) 212 of FIGS. 2A-2D), and a discussion of the drainage elements 310a-b will be limited to those features that different from the drainage element(s) 110 of FIG. 1 and/or the drainage element(s) 210 of FIGS. 2A-2D, and those features necessary for context. Additionally, any features of the drainage elements 310a-b can be combined with each other and/or the respective drainage element(s) 110, 210 of FIGS. 1 and/or 2A-2D.

Referring first to FIG. 3A, the drainage element 310a can include a projection or tab 311 extending from the interface surface 313a. The projection 311 can include the drainage inlet 312a, such that the drainage inlet 312a opening is generally or substantially perpendicular to the interface surface 313a. In such embodiments, the drainage inlet 312a can have a width W₄ greater than a width W₅ of the channel 316a. In at least some embodiments, for example, the drainage inlet 312a width W₄ can be between about 40 μm and about 150 μm, such as at least 40 μm, at least 50 μm, at least 60 μm, at least 70 μm, at least 80 μm, at least 90 μm, at least 100 μm, or any other suitable width; and the channel 316a width W₅ can be between about 5 μm and about 50 μm, such as up to 10 μm, up to 15 μm, up to 20 μm, up to 25 μm, up to 30 μm, up to 35 μm, up to 40 μm, or any other suitable width. Without being bound by theory, drainage elements including drainage inlets having openings that are perpendicular to the interface surface 313a and that have widths W₄ greater than the corresponding channels widths W₅ can allow fluid to enter the drainage inlets when the drainage inlets are positioned close to a corresponding gating element configured to selectively control the fluid flow through the inlet, such as described in detail below with reference to the gating element 512 of FIGS. 5A-6B and the gating element 702 of FIGS. 7A and 7B. For example, this can reduce the distance D the gating element must travel to move between a first (e.g., open) position permitting fluid to flow into the inlet 312 to a second (e.g., closed) position preventing or at least partially reducing fluid from flowing into the inlet 312 (as described below with reference to FIGS. 6A-6B). In at least some embodiments, for example, the distance D can be between about 5 μm and about 50 μm, such as up to 10 μm, up to 15 μm, up to 20 μm, up to 25 μm, up to 30 μm, up to 35 μm, up to 40 μm, or up to any other suitable distance.

The channels in the drainage elements described herein can take any suitable shape that permits the length of the channel to be greater than the height of the drainage element. For example, FIG. 3B illustrates a drainage element 3 10b including a channel 3 16b having a curved or spiral shape. In other embodiments, the channel 316b can have a zig-zag shape, a curvilinear shape, a combination of one or more of any of the channel shapes described herein, or any other suitable shape. In the illustrated embodiment, the lumen portion 314b is concentric with, or positioned along a longitudinal axis of, the drainage element 310b. In other embodiments, the lumen portion 314b can be positioned near a periphery of the drainage element 310b, or in any other suitable position in the drainage element 310b. In the illustrated embodiment, the lumen portion 314 has a circular shape. In other embodiments, the lumen portion 314 can have an oval shape, a curvilinear shape, triangular shape, a square shape, a rectangular shape, a rectilinear shape, a pentagonal shape, a hexagonal shape, or any other suitable shape.

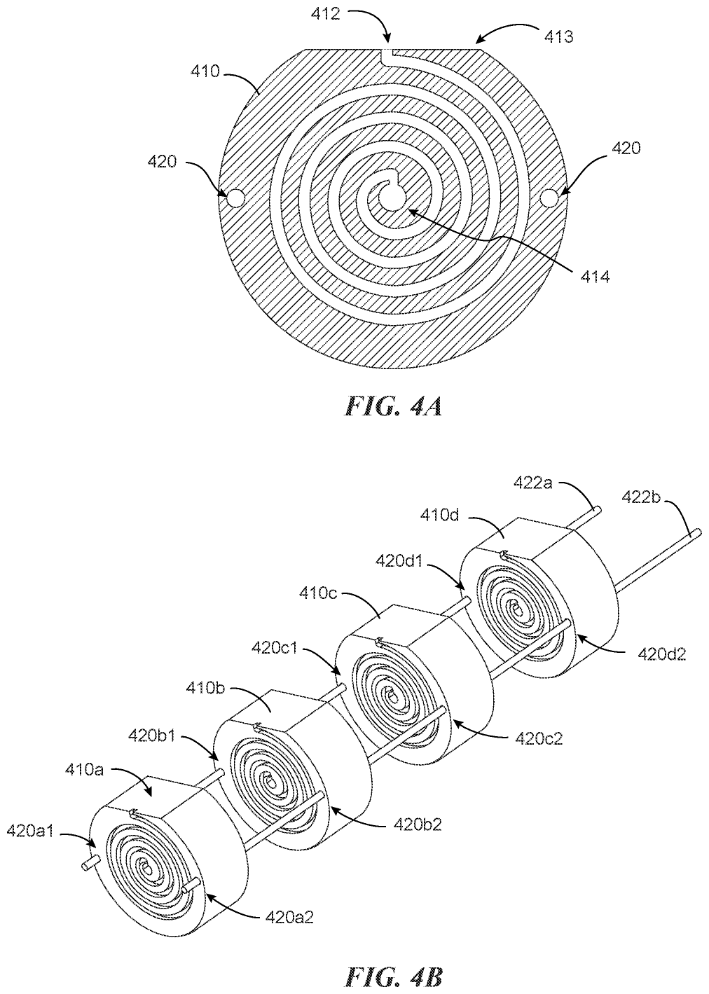

FIG. 4A illustrates another drainage element 410 configured in accordance with select embodiments of the present technology. The drainage element 410 and/or one or more components thereof can be generally similar or the same as the respective drainage element(s) 110, 210, 310 of FIGS. 1-3B. Accordingly, like numbers are used to indicate like components (e.g., drainage element 410 versus the respective drainage element(s) 110, 210, 310 of FIGS. 1-3B), and a discussion of the drainage element 410 of FIG. 4 will be limited to those features that differ from the respective drainage element(s) 110, 210, 310 of FIGS. 1-3B, and those features necessary for context. Additionally, any features of the drainage element 410 can be combined with the respective drainage elements 110, 210, 310 of FIGS. 1-3B.

As shown in FIG. 4A, the drainage element 410 can include one or more alignment apertures 420. Each of the alignment apertures 420 can be used to align or orient the drainage element 410 relative to one or more other drainage elements, to align or orient one or more first features (e.g., a first drainage inlet 412, a first drainage lumen portion 414, a first interface surface 413, etc.) of a first drainage element with one or more second features of a second drainage element (e.g., a second drainage inlet, a second drainage lumen portion, a second interface surface, etc.), and/or to align or orient the drainage element 410 relative to a carriage element (e.g., the carriage element 202 of FIGS. 2A and 2B).

FIG. 4B illustrates a plurality of the drainage elements 410a-d of FIG. 4B. Each of the drainage elements 410a-d can be aligned and/or oriented relative to each other by one or more alignment elements 422, e.g., to reduce or prevent movement or rotation of a first drainage element 410a relative to one or more other drainage elements 410b-d. In the illustrate embodiment, for example, each of the drainage elements 410a-d includes a respective first alignment aperture 420a₁, 420b₁, 420c₁, 420d₁ configured to receive a corresponding first alignment element 422a, and further includes a respective second alignment aperture 420a₂, 420b₂, 420c₂, 420d₂ configured to receive a corresponding second alignment element 422b.

Referring to FIGS. 4A and 4B together, in the illustrated embodiment, the alignment apertures 420 have a circular shape; in other embodiments each of the alignment apertures 420 can have an oval shape, a curvilinear shape, triangular shape, a square shape, a rectangular shape, a rectilinear shape, a pentagonal shape, a hexagonal shape, or any other suitable shape. In some embodiments, the first alignment apertures 420a₁, 420b₁, 420c₁, 420d₁ can have a first shape, and the second alignment apertures 420a₂, 420b₂, 420c₂, 420d₂ can have a second shape different than the first shape. Each of the alignment elements 422 can have a size and/or shape that corresponds to individual corresponding ones of the alignment apertures 420. In at least some embodiments, for example, the first alignment element 422a can have a first size and/or shape corresponding to a size and/or shape of the first alignment apertures 420a₁, 420b₁, 420c₁, 420d₁, and the second alignment element 422b can have a second size and/or shape corresponding to a size and/or shape of the second alignment apertures 420a₂, 420b₂, 420c₂, 420d₂. Although each drainage element 410 in FIGS. 4A and 4B are illustrated as having two alignment apertures 420, in other embodiments each drainage element 410 can include more or fewer alignment apertures 420. In at least some embodiments, for example, each drainage element 410 can include one, three, four, five, or more alignment apertures 420. In some embodiments, the alignment apertures 420 and the alignment elements 422 can be used to align, stack, couple, etc., two or more of the drainage elements 410 prior to insertion into a carriage assembly, such as the assembly 200 of FIGS. 2A-2C.

FIGS. 5A and 5B illustrate an actuation assembly 500 for selectively controlling fluid flow through the fluid inlets described herein and configured in accordance with select embodiments of the present technology. For example, the actuation assembly 500 can be used as part of the drainage assembly 100, 200 of FIGS. 1-2D, e.g., to selectively control the flow of fluid entering the drainage elements. Referring collectively to FIGS. 5A and 5B, the actuation assembly 500 can include a housing or frame 502 including and/or defining a plurality of chambers or wells 504. In the illustrated embodiment, for example, the housing 502 includes a first well 504a, a second well 504b, and a third well 504c. Each of the wells 504 can include one or more actuators 510. In the illustrated embodiment, for example, the first well 504a includes a first actuator 510a, the second well 504b includes a second actuator 510b, and the third well 504c includes a third actuator 510c. Each of the actuators 510 can include a control or gating element 512 coupled to one or more first actuation elements 514 and one or more second actuation elements 516. Additionally, the first actuation element(s) 514 can be coupled to the housing 502, and the second actuation element(s) 516 can be coupled to a priming and/or anchoring element 518 (“the priming element 518”). In at least some embodiments, the gating element 512, the first actuation element(s) 514, the second actuation element(s) 516, and/or the priming element 518 can be coplanar. The priming element 518 is discussed in greater detail below and with reference to FIG. 5B.

In the illustrated embodiment, the first actuator 510a includes a first gating element 512a coupled to three first actuation elements 514a₁₋₃ (e.g., struts) and three second actuation elements 516a₁₋₃, the second actuator 510b includes a second gating element 512b coupled to three first actuation elements 514b 1-3 and three second actuation elements 516b₁₋₃, and the third actuator 510c includes a third gating element 512c coupled to three first actuation elements 514c₁₋₃ and three second actuation elements 516c₁₋₃. Individual ones of the first actuation elements 514 and/or the second actuation elements 516 can be arranged generally parallel to each other when in their preferred geometry. In other embodiments, the actuation assembly 500 can include more or fewer wells 504, actuators 510, first actuation elements 514, and/or second actuation elements 516. In at least some embodiments, for example, the actuation assembly 500 can include one, two, four, five, six, seven, eight, nine, ten, or any other suitable number of chambers 504, actuators 510, first actuation elements 514, and/or second actuation elements 516 (e.g., to correspond to the number of drainage elements and/or fluid inlets on a corresponding drainage assembly). In at least some embodiments, each of the actuators 510 can include a first number of first actuation elements 514 and a second number of second actuation elements 516 the same as or different than the first number of first actuation elements 514.

The actuation assembly 500 can be configured to selectively control the flow of fluid entering an adjustable shunting system, such as any of the systems described previously and/or incorporated by reference herein. In particular, the first actuator 510a can be configured to control the flow of fluid through a first fluid inlet (e.g., the first drainage inlet 112a of FIG. 1), the second actuator 510b can be configured to control the flow of fluid through a second fluid inlet (e.g., the second drainage inlet 112b of FIG. 1), and the third actuator 510c can be configured to control the flow of fluid through a third fluid inlet (e.g., the third drainage inlet 112c of FIG. 1). Each of the projection or gating element(s) 512 can be configured to moveably interface with a corresponding fluid inlet, e.g., to move between a first (e.g., “open”) position in which the gating element 512 does not substantially prevent fluid from flowing through the corresponding fluid inlet (e.g., by not interfering with the corresponding fluid inlet) and a second (e.g., “closed”) position in which the gating element 512 substantially prevents fluid from flowing through the corresponding fluid inlet (e.g., by blocking the corresponding fluid inlet). In some embodiments, the gating element(s) 512 can be configured to move to one or more intermediate positions between the first (e.g., open) and the second (e.g., closed) position.

The first actuation element(s) 514 and the second actuation element(s) 516 can drive movement of the gating element(s) 512 between the first (e.g., open) position and the second (e.g., closed) position. The first actuation element(s) 514 and the second actuation element(s) 516 can be composed at least partially of a shape memory material or alloy (e.g., nitinol). Accordingly, the first actuation element(s) 514 and the second actuation element(s) 516 can be transitionable at least between a first material phase or state (e.g., a martensitic state, a R-phase, a composite state between martensitic and R-phase, etc.) and a second material phase or state (e.g., an austenitic state, an R-phase state, a composite state between austenitic and R-phase, etc.). In the first material state, the first actuation element(s) 514 and the second actuation element(s) 516 may have reduced (e.g., relatively less stiff) mechanical properties that cause the actuation elements to be more easily deformable (e.g., compressible, expandable, etc.). In the second material state, the first actuation element(s) 514 and the second actuation element(s) 516 may have increased (e.g., relatively more stiff) mechanical properties relative to the first material state, causing an increased preference toward a specific preferred geometry (e.g., original geometry, manufactured or fabricated geometry, heat set geometry, etc.). The first actuation element(s) 514 and the second actuation element(s) 516 can be selectively and independently transitioned between the first material state and the second material state by applying energy (e.g., laser energy, electrical energy, etc.) to the first actuation element(s) 514 or the second actuation element(s) 516 to heat it above a transition temperature (e.g., above an austenite finish (Ar) temperature, which is generally greater than body temperature). If the first actuation element(s) 514 (or the second actuation element(s) 516) is deformed relative to its preferred geometry when heated above the transition temperature, the first actuation element(s) 514 (or the second actuation element(s) 516) will move to and/or toward its preferred geometry.

The first actuation element(s) 514 and the second actuation element(s) 516 generally act in opposition. For example, the first actuation element(s) 514 can be actuated to move the corresponding gating element(s) 512 to and/or toward the first (e.g., open) position, and the second actuation element(s) 516 can be actuated to move the corresponding gating element(s) 512 to and/or toward the second (e.g., closed) position. Additionally, the first actuation element(s) 514 and the second actuation element(s) 516 can move in concert, such that as one moves toward its preferred geometry upon material phase transition, the other is deformed relative to its preferred geometry. This enables the actuation elements 514, 516 to be repeatedly actuated and the gating element(s) 512 to be repeatedly cycled between the first (e.g., open) position and the second (e.g., closed) position.

In the embodiment illustrated in FIG. 5A, the actuation elements 514, 516 are in their preferred (e.g., as-manufactured) geometry, and the gating element is in the first (e.g., open) position. Referring next to FIG. 5B, the priming element(s) 518 can be moved to contact and/or be coupled to a priming surface 506 of the housing 502. In some embodiments, coupling the priming element(s) 518 to the priming surface 506 does not substantially move the gating element(s) 512 (e.g., the gating element(s) 512 can remain in the first position, as shown in FIGS. 5A and 5B). In other embodiments, coupling the priming element(s) 518 to the priming surface(s) 506 causes the gating element(s) 512 to move to an intermediate position between the first position and the second position. Regardless, coupling the priming element(s) 518 to the priming surface(s) 506 can deform/strain the second actuation element(s) 516 relative to their preferred geometry (FIG. 5A) such that the second actuation element(s) 516 are stressed.

Once in the stressed configuration shown in FIG. 5B, the second actuation element(s) 516 can be actuated to move the gating element(s) 512 toward the second position. For example, applying energy to the second actuation element(s) 516 heats the second actuation element(s) above its transition temperature, which causes the second actuation element(s) to transition from the relatively less stiff first material state to the relatively stiffer second material state. This also causes the second actuation element(s) to move toward its preferred geometry, reducing the stress within the second actuation elements. For example, the second actuation elements 516 can return toward their linear (e.g., non-warped, non-stressed) configuration (i.e., their preferred geometry). However, because the second actuation elements 516 are coupled to the frame 502 via the priming element 518, the second actuation elements 516 do not return to the position shown in FIG. 5A. Rather, the second actuation elements 516 move at least partially away from the first actuation elements 514 and toward the frame 502. Because the gating element(s) 512 are coupled to the second actuation element(s) 516, movement of the second actuation element(s) 516 toward their preferred geometry causes the gating element to translate (e.g., slide) within the well toward the second actuation element(s) 516 and the second (e.g., closed) position. This movement deforms the first actuation element(s) 514. As described previously, this enables the actuation elements 514, 516 to be repeatedly actuated and the gating element(s) 512 to be repeatedly cycled between the first (e.g., open) position and the second (e.g., closed) position (not shown).

In the illustrated embodiment, individual ones of the first actuation elements 514 and the second actuation elements 516 have a generally tapered or hourglass shape. Actuation elements 514, 516 that are tapered or hourglass shaped can have a relatively more consistent strain and/or stress distribution (e.g., across the length and/or width of the individual actuation elements 514, 516) when deformed relative to their preferred geometries. Relatively consistent strain across an actuation element can enable greater overall movement of the actuation element and/or the gating element to which the actuation element is coupled, without bringing the material (e.g., the material forming the actuation element) to a material limit. For example, the material limit can be a strain limit, beyond which the material is no longer thermo-elastically recoverable and, as a result, unable to reliably or consistently move between the first and second positions. Thus, without being bound by theory, it is believed that actuation elements 514, 516 configured in accordance with embodiments of the present technology can improve the motion (e.g., consistency, linearity, etc.) of the gating element(s) 512 between the first and second positions. In other embodiments, individual ones of the first actuation element(s) 514 and/or the second actuation element(s) 516 can have a linear shape, a rectilinear shape, a curvilinear shape, a zig-zag shape, a serpentine shape, or any other suitable shape.

In some embodiments, the gating element(s) 512 can include one or more end portions. As best seen in FIG. 5A, for example, the first actuator 510a can include a first end portion 513a₁ coupled to the first actuation element(s) 514a and a second end portion 513a₂ coupled to the second actuation element(s) 516b. The first gating element 512a can be positioned between and coupled to the first and second end portions 513a₁₋₂. In some embodiments, the first end portion 513a₁ the second end portion 513a₂ can be on and/or extend from opposite sides of the gating element 512a. In some embodiments, the first end portion 513a₁ and/or the second end portion 513a₂ can be generally or substantially perpendicular to the gating element 512a and/or a longitudinal axis of the housing 502. In some embodiments, the first actuation element(s) 514 and the second actuation element(s) 516 can be selectively actuated without or substantially without deforming or torqueing the first end portion 513a₁ and/or the second end portion 513a₂ relative to each other or the first gating element 512a. Accordingly, the motion of the gating element 512a between the first and second positions can be generally or substantially linear, e.g., generally or substantially perpendicular to and/or in a lateral direction relative to a longitudinal axis of the housing 502.

In some embodiments, energy can be applied to the one or more end portions 513 to actuate individual ones of the first actuation element(s) 514 and/or the second actuation element(s) 516. The energy applied to the one or more end portions 513 may spread into and therefore heat the first actuation element(s) 514 and/or the second actuation element(s) 516. In at least some embodiments, for example, energy can be applied to the first end portion 513a₁ to actuate one or more of the first actuation elements 514a₁₋₃, and/or energy can be applied to the second end portion 513a₂ to actuate one or more of the second actuation elements 516a₁₋₃, e.g, to move the first gating element 512a between the first and second positions as described previously. Energy can also be applied to the end portion(s) of the second actuator 510b and/or the third actuator 510c. Without being bound by theory, applying energy to the one or more end portions 513 is expected to simplify the actuation process due to the relatively larger surface area (and thus relatively larger target) that energy can be applied to. However, in some embodiments, energy can be applied directly to the first actuation element(s) 514 and/or to the second actuation element(s) 516.

Referring to FIGS. 5A and 5B together, in some embodiments, the actuation assembly 500 can be a unitary or contiguous structure (e.g., cut from, printed as, or deposited as a single piece of material). For example, each of the actuators 510 can be patterned (e.g., cut, laser cut, formed, etc.) in a single piece of material (e.g., nitinol), and the well(s) 504 can correspond to regions of the single piece of material that were removed (e.g., during a subtractive manufacturing process) or where material was not added (e.g., during an additive manufacturing process). Additional details regarding the operation and manufacture of shape memory actuators, as well as adjustable glaucoma shunts, are described in U.S. Pat. No. 11,291,585, U.S. Pat. No. 11,166,849, and International Patent Application Nos. PCT/US20/55144, PCT/US20/55141, PCT/US21/14774, PCT/US21/18601, PCT/US21/023238, and PCT/US21/27742, the disclosures of which are incorporated by reference herein in their entireties and for all purposes.

FIGS. 6A and 6B are tops views of a flow control system 600 including the drainage assembly 100 of FIG. 1 and the actuation assembly 500 of FIGS. 6A and 6B. Referring to FIGS. 6A and 6B together, as described previously, the actuation assembly 500 can be used to control the flow of fluid through the drainage inlets 112. In the illustrated embodiment, for example, the first gating element 512a is configured to control the flow of fluid through the third drainage inlet 112c, the second gating element 512b is configured to control the flow of fluid through the second drainage inlet 112b, and the third gating element 512c is configured to control the flow of fluid through the first drainage inlet 112a. As best seen in FIG. 6B, the actuation assembly 500 can be positioned in the slot 106 of the carriage element 102 and/or coupled to the drainage element(s) 110, e.g., coupled to the interface surface(s) 113 of the drainage element(s) 110). Although the flow control system 600 illustrated in FIGS. 6A and 6B includes the drainage assembly 100 of FIG. 1 and the actuation assembly 500 of FIGS. 5A and 5B, in other embodiments the flow control system 600 can include any combination of the actuation assemblies and/or drainage assemblies described herein and/or incorporated by reference herein.

FIGS. 7A and 7B illustrate another actuator 710 for controlling the flow of fluid in a shunting system and configured in accordance with select embodiments of the present technology. More specifically, FIG. 7A is an isometric view of the actuator 710 in a fabricated or non-tensioned configuration, and FIG. 7B is a top view of the actuator 710 in the fabricated or non-tensioned configuration. The actuator 710 is shown in isolation for clarity. However, as one skilled in the art will appreciate, the actuator 710 can be used to selectively control the flow of fluid through inlets of the assembly 100 and/or the assembly 200 shown in FIGS. 1-2D, (e.g, in lieu of the actuation assembly 500 shown in FIGS. 5A-6B).

The actuator 710 can include a first shape-memory actuation element 714, a second shape memory actuation element 716, and a gating element 702 coupled to and positioned between the first shape-memory actuation element 714 and the second shape memory actuation element 716. When the actuation elements 714, 716 are deformed relative to their preferred (e.g, as manufactured) geometries, applying energy to the actuation elements 714, 716 (e.g., via respective targets 724a, 724b) induces a material phase change in the actuation element that causes the actuation element to transition toward its preferred geometry, similar to the transitioning described previously with respect to the actuation elements 514, 516 in FIGS. 5A and 5B. This transition imparts a rotational movement in the gating elements 702. The gating element 702 can therefore be moved between a first position blocking a fluid inlet (e.g., the drainage inlet(s) 212, shown in FIGS. 2A-2D) and a second position in which the gating element 702 does not interfere with a fluid inlet. The actuator 710 is described in greater detail in U.S. patent application Ser. No. 17/175,332, previously incorporated by reference herein.

As one skilled in the art will appreciate, any of the actuation assemblies and/or actuators described above can be used with the assembly 100 or the assembly 200 of respective FIG. 1 and FIGS. 2A-2D to control the flow of fluid therethrough. Moreover, certain features described with respect to one assembly and/or drainage element can be added or combined with another assembly and/or drainage element. Accordingly, the present technology is not limited to the assemblies and/or drainage elements expressly identified herein.

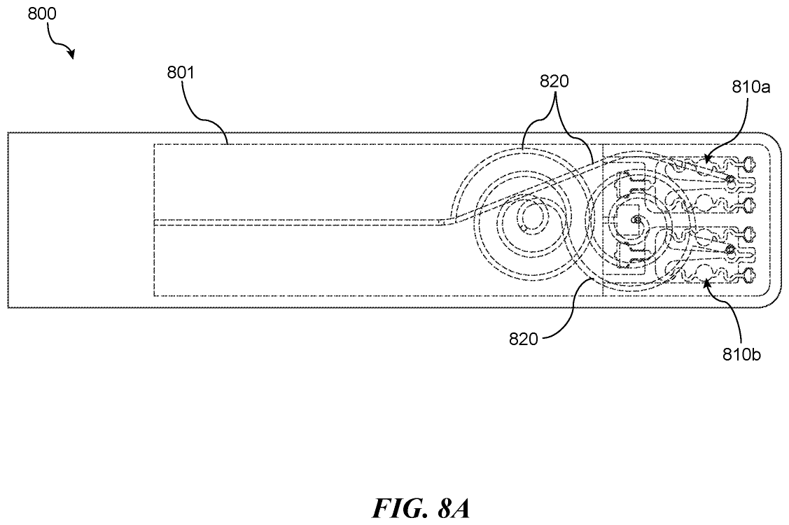

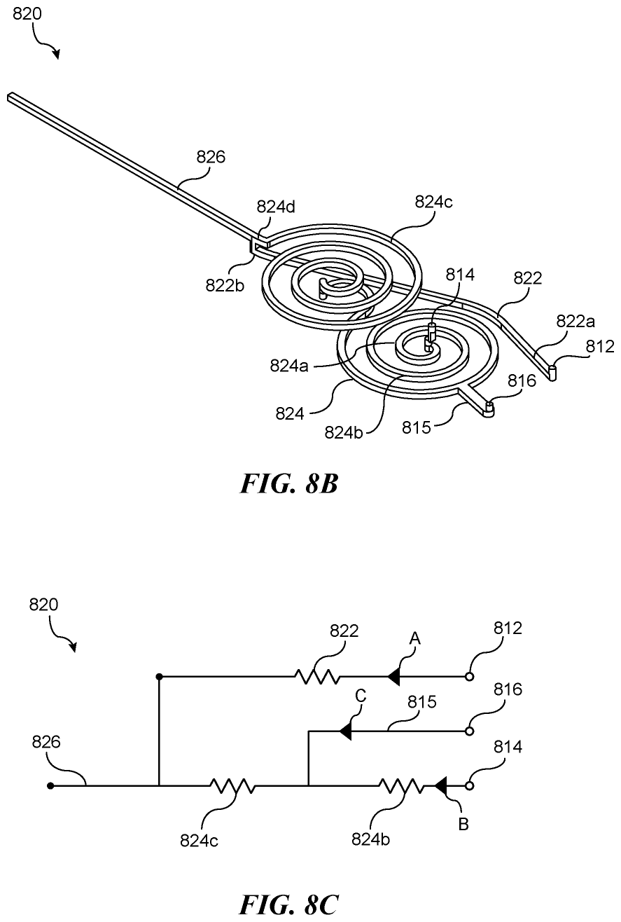

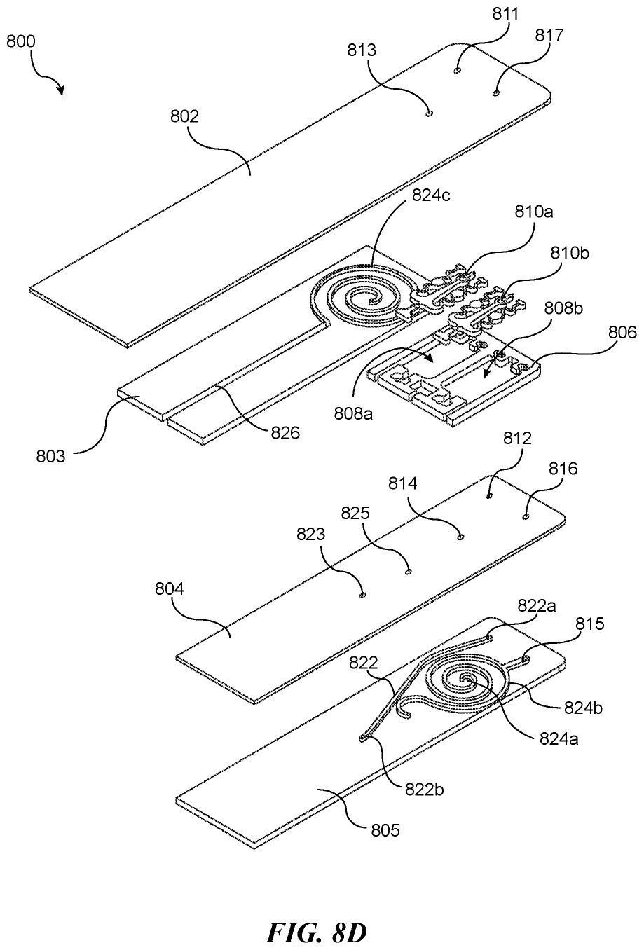

FIGS. 8A-8D illustrate a shunting system 800 (“the system 800”) configured in accordance with select embodiments of the present technology. More specifically, FIG. 8A is a top-down view of the system 800, FIG. 8B is a perspective view of a fluid resistor network 820 extending through the system 800 with other aspects of the system 800 omitted for clarity, FIG. 8C is a schematic representation of the fluidic resistor network 820 shown in FIG. 8B, and FIG. 8D is an exploded isometric view of the system 800. As described in greater detail below, the system 800 is configured to provide a titratable therapy for draining fluid from a first body region to a second body region, such as to drain aqueous from an anterior chamber of a patient's eye to aid in the treatment of glaucoma.

Referring to FIG. 8A, the system 800 includes an elongated housing or shunting element 801 defining the network of fluid resistors 820 (also referred to herein as the “fluid resistor network 820”) and housing one or more actuators 810 (shown as a first actuator 810a and a second actuator 810b). As described in greater detail with reference to FIGS. 8B-8D, the network of fluid resistors 820 can comprise a plurality of channels or lumens through which fluid can flow through the shunting element 801 when the system 800 is implanted within a patient. As also described in greater detail with reference to FIGS. 8B-8D, the actuators 810 can selectively control the flow of fluid through the fluid resistor network 820, such as by selectively interfering with and/or selectively not interfering with associated fluid flow ports. In some embodiments, the actuators are shape memory actuators, such as those described above with reference to FIGS. 5A, 5B, 7A, and 7B, and/or those described in U.S. Patent Application Publication Nos. US 2020/0229982 and US 2021/0251806, the disclosures of which are incorporated by reference herein in their entireties and for all purposes.

FIG. 8B illustrates the network of fluid resistors 820 with other aspects of the system 800 omitted for purposes of illustration and clarity. The network of fluid resistors 820 can include a plurality of at least partially isolated channels or lumens. For example, in the illustrated embodiment, the network of fluid resistors 820 includes a first channel 822 and a second channel 824, which merge to form a third channel 826. The first channel 822 includes a first end portion 822a and a second end portion 822b. The first end portion 822a is aligned with (e.g., fluidly coupled to) a corresponding first fluid flow port 812 (“the first port 812”), and the second end portion 822b is fluidly coupled to the third channel 826. As illustrated, the first channel 822 has a relatively linear or straight flow path. Accordingly, the first channel 822 and the third channel 826 can form a first flow path through the system 800.

The second channel 824 includes a first end portion 824a, a first spiral or portion or segment 824b, a second spiral portion or segment 824c, and a second end portion 824d. The first end portion 824a is fluidly aligned with (e.g., fluidly couped to) a corresponding second fluid flow port 814 (“the second port 814”), and the second end portion 824d is fluidly coupled to the third channel 826. The first spiral portion 824b extends between and fluidly couples the first end portion 824a and the second spiral portion 824c. The second spiral portion 824c extends between and fluidly couples the first spiral portion 824b and the second end portion 824d. The second channel 824 can further include a bypass channel 815 fluidly coupled to a portion of the first spiral portion 824b. The bypass channel 815 can extend from a corresponding third fluid flow port 816 (“the third port 816”). Accordingly, the second channel 824 defines two additional flow paths through the system 800: (1) a first flow path beginning at the second port 814, through the full length of the second channel 824, and through the third channel 826, and (2) a second flow path beginning at the third port 816, through the bypass channel 815, through a portion of the second channel 824, and through the third channel 826.

Each of the channels and/or flow paths of the network of fluid resistors 820 can be associated with a different fluid resistance. For example, for a given cross-sectional area, the fluid resistance provided by a channel is proportional to the length of the channel. That is, a longer channel tends to have a higher fluid resistance, and a shorter channel tends to have a lower fluid resistance. Accordingly, in embodiments in which the first channel 822 and the second channel 824 have the same or substantially the same cross-sectional dimension (e.g., diameter), the first channel 822 has a lower fluid resistance than the second channel 824. This is because the second channel 824, by virtue of the first spiral portion 824b and the second spiral portion 824c, has a greater length than the first channel 822. Similarly, the fluid resistance of the flow pathway extending between the third port 816 and the third channel 826 is less than the fluid resistance of the flow pathway extending between the second port 814 and the third channel 826 because the length of the flow pathway between the third port 816 and the third channel 826 is less than the length of the flow pathway between the second port 814 and the third channel 826. In some embodiments, the third channel 826 has a cross-sectional dimension (e.g., diameter) substantially greater than the corresponding cross-sectional dimensions of the first channel 822 and the second channel 824, such that the third channel 826 has a negligible impact on the fluid resistance through the various flow pathways. Similarly, the bypass channel 815 and the various ports can also have a negligible impact on the fluid resistance through the various flow pathways. As one skilled in the art will appreciate, resistance is more sensitive to changes in cross-sectional area than length, and so the third channel 826, the bypass channel 815, and the various ports can be designed to have a negligible impact on the overall flow resistance simply by having a slightly larger cross-sectional area than the first channel 922 and the second channel 824. In such embodiments, the lengths and cross-sectional areas of the first channel 822 and second channel 824 can be designed to impart a desired fluid resistance. Of course, in other embodiments the third channel 826, the bypass channel 815, and/or the various ports can impart a meaningful fluid resistance to the various flow pathways.

FIG. 8C is a schematic diagram of the fluid resistor network 820 further demonstrating the relative fluid resistance of the various flow pathways through the fluid resistor network 820. As illustrated, the first channel 822, the first spiral portion 824b, and the second spiral portion 824c are the primary sources of resistance through the network 820. That is, the ports 812, 814, 816, the bypass channel 815, and the third channel 826 do not impart a meaningful resistance through the network 820. As a result, the network 820 provides three fluid flow paths through the system: a first flow path A between the first port 812 and the third channel 826 via the first channel 822, a second flow path B between the second port 814 and the third channel 826 via the first spiral portion 824b and the second spiral portion 824c, and a third flow path C between the third port 816 and the third channel 826 via the bypass channel 815 and the second spiral portion 824c. In the illustrated embodiment, the first flow path A has the lowest resistance, the second flow path B has the highest resistance, and the third flow path C has an intermediate resistance between the first resistance and the second resistance. As one skilled in the art will appreciate from the disclosure herein, the channels can be arranged to provide different relative resistances than those depicted herein. Accordingly, the present technology is not limited to any particular configuration of channels, unless expressly noted otherwise. The fluid resistor networks can also have more or fewer channels. Indeed, additional examples of fluid resistor networks are described in Section C below.

The system 800 is composed of multiple layers of material stacked and sealingly coupled (e.g., adhered) together. For example, FIG. 8D is an exploded isometric view of the system 800 showing a plurality of layers (shown as a first layer 802, a second layer 803, a third layer 804, and a fourth layer 805). The first layer 802, the second layer 803, the third layer 804, and the fourth layer 805 can be adhered together to form the system 800 using any suitable manufacturing process, including welding, gluing, chemical bonding, or other suitable techniques, as described in greater detail under Section B. Once adhered, adjacent surfaces of the first layer 802, the second layer 803, the third layer 804, and the fourth layer 805 form a fluidic and airtight seal such that fluid or gas cannot leak between adjacent layers (other than through defined flow pathways, such as the fluid resistor network 820). Once assembled together, each of the layers 802-805 forms a plane that is parallel to planes formed by each of the other layers 802-805, and to the longitudinal axis of the system 100. This is in contrast to the stackable drainage elements described above with reference to FIGS. 2A-4B in which each stackable drainage element forms a plane perpendicular to the longitudinal axis of the corresponding system.

The layers 802-805 can be composed of the same material or different materials. For example, in some embodiments one or more of the first layer 802, the second layer 803, the third layer 804, and/or the fourth layer 805 can be composed of silicone, plastic, glass, a polymer, or another suitable material. As described in greater detail below, the ability to manufacture individual layers out of different materials is expected to be beneficial because different layers can be manufactured to have different material properties (e.g., different levels of rigidity) that are based on (e.g., optimized to) the function of the layer.

Each of the layers 802-805 can be a flat layer having a width-to-height or width-to-thickness ratio of greater than about 10:1 or greater than about 50:1, such as between about 10:1 and about 100:1. The layers 802-805 can have the same or different height/thicknesses. For example, in some embodiments one or more of the first layer 802, the second layer 803, the third layer 804, and/or the fourth layer 805 have a thickness between about 10 μm and about 500 μm, or between about 10 μm and about 100 μm, or between about 10 μm and about 50 μm. In some embodiments, the first layer 802, the second layer 803, the third layer 804, and/or the fourth layer 805 can have thickness less than about 500 μm, less than about 250 μm, less than about 100 μm, less than about 50 μm, and/or less than about 25 μm. Without being bound by theory, manufacturing the layers 802-805 to have a relatively high width-to-thickness ratio (e.g., greater than 10:1) and a relatively small thickness (e.g., less than about 500 μm) will advantageously result in the overall footprint of the system 300 being relatively compact.

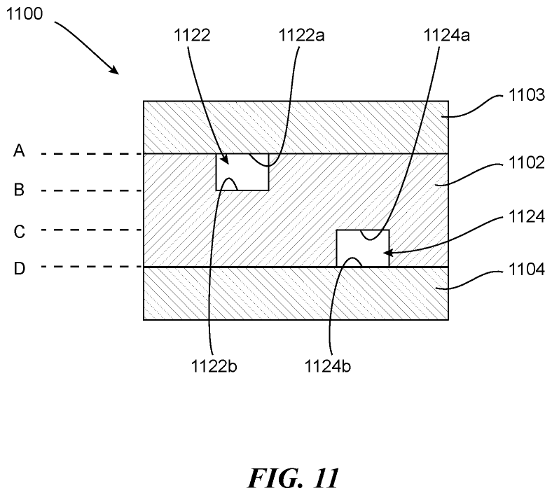

The fluid resistor network 820 can occupy multiple layers of the system 800. In the illustrated embodiment, for example, the first channel 822 and the first spiral portion 824b of the second channel 824 are at least partially defined within the fourth layer 805, and the second spiral portion 824c of the second channel 824 and the third channel 826 are at least partially defined within the second layer 803. That is, the first channel 822 and the first spiral portion 824b are formed in a first plane parallel to the longitudinal axis of the system, and the second spiral portion 824c and the third channel 826 are formed in a second, overlapping plane that is parallel to the first plane and the longitudinal axis of the system. As used herein, “at least partially defined” when used in the context of describing a channel within a layer of material includes embodiments in which at least 80%, at least 90%, or at least 95% of the void space of the channel is defined within the layer of material. In some embodiments, the layer that “at least partially defines” a channel does not fully enclose the channel itself. Rather, another layer may form the “top” or “bottom” surface of the channel, such that the channel only becomes enclosed when the two adjacent layers are adhered together. An example of such embodiment is described in greater detail below with reference to FIG. 11. In other embodiments, the channel, including the walls defining the channel, are all formed within a single layer.

The first layer 802 (e.g., the top layer) can include a first aperture 811, a second aperture 813, and a third aperture 817. As described below, the first aperture 811, the second aperture 813, and the third aperture 817 can permit fluid to flow into the system 800, and can therefore be described as first, second, and third inlets, respectively. The third layer 804 can also include various ports or apertures that permit fluid to enter the fluid resistor network 820 and/or flow between various channels of the fluid resistor network 820. For example, the third layer 804 includes the first port 812 that provides fluid access to (e.g., is aligned with) the first end portion 822a of the first channel 822, the second port 814 that provides fluid access to (e.g., is aligned with) the first end portion 824a of the second channel 824, and the third port 816 that provides fluid access to (e.g., is aligned with) the bypass channel 815. The first port 812, the second port 814, and the third port 816 are positioned to be in fluid communication with the first aperture 811, the second aperture 813, and the third aperture 817, respectively, in the first layer 802. As described below, this enables fluid to flow into the fluid resistor network 820 via the first aperture 811, the second aperture 813, and the third aperture 817.

The third layer 804 further includes various apertures that serve as portals or connectors between portions of the fluid resistor network 820 positioned in different layers. For example, the third layer 804 includes a first connector 823 and a second connector 825. The first connector 823 and the second connector 825 can be through-holes, lumens, ports, or the like that extend between an upper surface and a lower surface of the third layer 804. Accordingly, the connectors 823, 825 generally have an axis that is perpendicular to the axis of the first channel 822, the second channel 824, and the third channel 826. The first connector 823 is positioned to extend between the second end portion 822b of the first channel 822 and the third channel 826 such that fluid flowing through the first channel 822 can flow into the third channel 826 via the first connector 823. The second connector 825 is positioned to extend between the first spiral portion 824b of the second channel 824 and the second spiral portion 824c of the second channel 824 such that fluid flowing the first spiral portion 824b can flow into the second spiral portion 824c.

Defining the fluid resistor network 820 in multiple layers of the system 800 is expected to provide several advantages. For example, defining the fluid resistor network 820 in multiple overlapping layers provides more volume in which the channels of the network 820 can be formed. As a result, channels of a particular resistance can be generally longer and wider than channels of the same resistance that are formed within a single layer of material (e.g., channel resistance is proportional to channel length and inversely proportional, by a function of the 4th power, to the channel diameter and so a first channel that is longer and wider than a second channel can have the same overall resistance as the second channel). This is expected to be advantageous for at least two reasons: (1) utilizing relatively wider/taller channels reduces the likelihood that cellular material or other debris will become stuck in, and therefore block, the channel, and (2) it is generally easier to manufacture wider channels than narrower channels, and any manufacturing variability in the diameter of the channel will have less impact on fluid resistance when the channel is wider. The foregoing advantages are particularly useful in embodiments in which the system 800 is small (e.g., embodiments in which the system 800 is a glaucoma shunt), and in which it may not be feasible to define a fluid resistor network 820 with suitable properties in a single layer.

As set forth above, the system 800 can also be selectively adjustable to provide a titratable therapy. For example, as shown in FIG. 8D, the system 800 can include the first actuator 810a, the second actuator 810b, and an actuator housing 806. The actuator housing 806 can include a first actuator chamber 808a configured to receive the first actuator 810a and a second actuator chamber 808b configured to receive the second actuator 810b. The actuator housing 806 can be composed of a material having a higher durometer (e.g., is more rigid) than the layers 802-805 so that the actuator housing 806 is resistant to deformation. The first actuator 810a can be configured to control the flow of fluid through the first port 812, and thus control the flow of fluid into the first channel 822. For example, the first actuator 810a can be moveable between at least a first position in which the first actuator 810a blocks the first port 812 and a second position in which the first actuator 810a does not block the first port 812. Likewise, the second actuator 810b can be configured to control the flow of fluid through the second port 816, and thus control the flow of fluid into the bypass channel 815, by moving between two or more positions. As set forth above, the first actuator 810a and the second actuator 810b can be composed of a shape memory material such as Nitinol, and can operate in the same or similar fashion as the actuators described above with reference to FIGS. 5A, 5B, 7A, and 7B, and/or the shape memory actuators described in U.S. Patent Application Publication Nos. US 2020/0229982 and US 2021/0251806, previously incorporated by reference herein. Although described as having two actuators 810a, 810b, one skilled in the art will appreciate that the system 800 can have more or fewer actuators, such as zero, one, three, four or more. Moreover, in some embodiments, each of the first port 812, the second port 814, and the third port 816 can include a corresponding actuator for controlling the flow of fluid therethrough. In other embodiments, such as the arrangement illustrated in FIG. 8D, at least one of the first port 812, the second port 814, and the third port 816 does not have a corresponding actuator, such that the at least one of the first port 812, the second port 814, or the third port 816 is constantly open to permit fluid to enter the fluid resistor network 820.

### B. Manufacturing Multi-Layered Resistors

The present technology further includes systems and methods expected to improve the manufacturing process of microfluidic shunting systems. As previously described with respect to FIGS. 8A-8D, the present technology includes shunting systems that are composed of a plurality of stackable layers (e.g., the layers 802-805 of the system 800). The layers 802-805 can be manufactured using the same or different process. For example, one or more of the first layer 802, the second layer 803, the third layer 804, and/or the fourth layer 805 can be manufactured via photolithography, spin casting, injection molding, laser cutting, or other suitable techniques. In some embodiments, the manufacturing technique used is based on one or more desired properties of the layers. For example, the “external” facing layers (i.e., the first layer 802 and the fourth layer 805) may be manufactured in a manner that produces an atraumatic and/or biocompatible surface. The “internal” layers (i.e., the second layer 803 and the third layer 804) may be manufactured in a manner that prioritizes other manufacturing properties (e.g, precision, tolerance, etc.).

Once the individual layers 802-805 are manufactured, the layers 802-805 can be stacked and adhered in the desired orientation. For example, as best shown in FIG. 8D, a second (e.g., “lower”) surface of the first layer 802 can be adhered to a first (e.g., “upper”) surface of the second layer 803, a second (e.g., “lower”) surface of the second layer 803 can be adhered to a first (e.g., “upper”) surface of the third layer 804, and a second (e.g., “lower”) surface of the third layer 804 can be adhered to a first (e.g., “upper”) surface of the fourth layer 805. As used herein, the terms “upper” and “lower” are used to specify surfaces of the layers 802-805 as shown in FIG. 8D. One skilled in the art will appreciate that, depending on the orientation of the system 800, an “upper” surface may be positioned below a “lower” surface, depending on the orientation of the system 800. Accordingly, the use of the term “upper” and “lower” do not require a specific spatial orientation relative to the ground, and are instead used for clarity of description. As set forth above in Section A, the individual layers 802-805 can be adhered using any suitable technique, such as gluing, bonding, taping, welding, soldering, stapling, suturing or the like. Non-limiting examples include self-adhesive or self-bonding via plasma treatment, via polymerization precursor material (e.g., use of uncured silicone to bond two adjacent layers of silicone together), and/or via ultrasonic bonding.

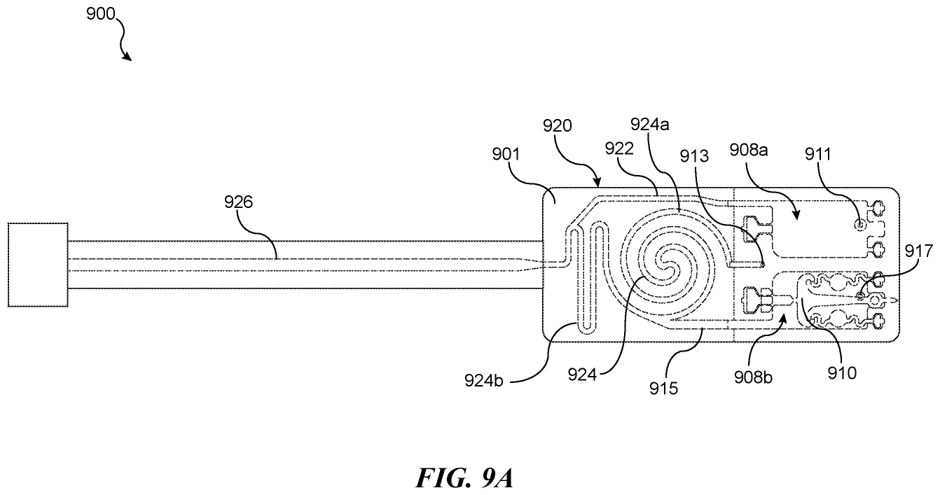

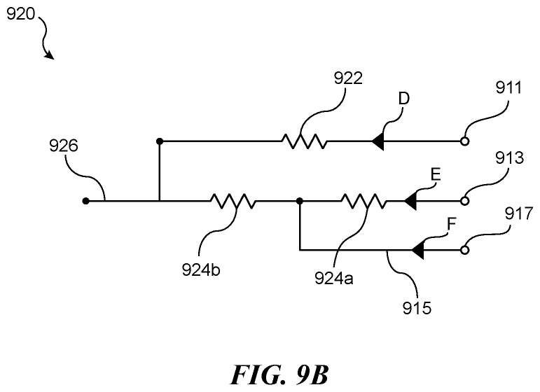

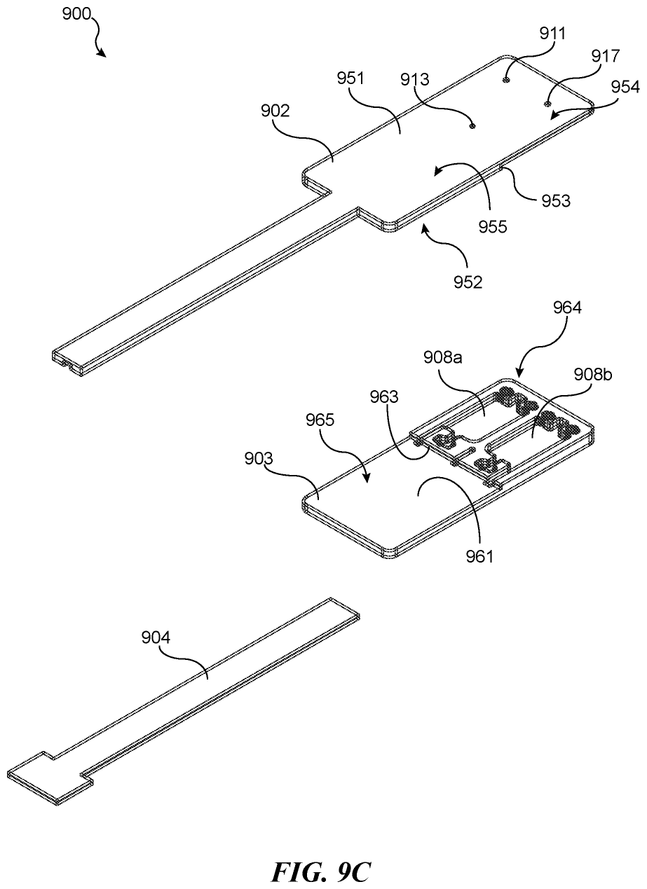

The shunting systems described herein can also have specific features expected to improve the manufacturability of the systems. For example, FIGS. 9A-9C illustrate a shunting system 900 (“the system 900”) configured in accordance with select embodiments of the present technology. More specifically, FIG. 9A is a top view of the system 900, FIG. 9B is a schematic representation of the fluidic resistor network shown in FIG. 9A, and FIG. 9C is an exploded isometric view of the system 900. Similar to the system 800 described previously, the system 900 can be an adjustable shunt configured to drain fluid from a first body region to a second body region, such as to drain aqueous from an anterior chamber of a patient's eye.

The system 900 can include certain features generally similar to certain features of the system 800 described with reference to FIGS. 8A-8D. For example, referring first to FIG. 9A, the system 900 includes an elongated housing or shunting element 901 defining a network of fluid resistors 920 (also referred to as the “fluid resistor network 920”). The fluid resistor network 920 can include a plurality of channels or lumens extending through the shunting element 901. For example, the system 900 includes a first channel 922, a second channel 924 having a first (e.g., spiral) portion or segment 924a and a second (e.g., serpentine) portion or segment 924b, and a third channel 926. The first channel 922, the second channel 924, and the third channel 926 can have an arrangement generally similar to the first channel 822, the second channel 824, and the third channel 826 described with reference to FIGS. 8A-8D. For example, the first channel 922 can extend between or otherwise fluidly connect a first port 911 (e.g., a first inlet aperture) and the third channel 926, while the second channel can extend between or otherwise fluidly connect a second port 913 (e.g., a second inlet aperture) and the third channel 926. The first channel 922 can have a different resistance than the second channel 924 by virtue of having a different length and/or cross-section. For example, in the illustrated embodiment the second channel 924 is substantially longer than the first channel 922 by virtue of its spiral and serpentine shape, and therefore has a higher resistance than the first channel 922. The third channel 926 can have a resistance less than the resistance of either the first channel 922 or the second channel 924. The system 900 can further include a bypass channel 915 extending between or fluidly coupling a third port 917 (e.g., a third inlet aperture) and a medial portion of the second channel 924 such that fluid entering the system 900 via the third port 917 bypasses the first (e.g, spiral) portion 924a of the second channel 924.

Similar to the system 800, the shunting element 901 can also define one or more actuator chambers (shown as a first actuator chamber 908a and a second actuator chamber 908b) for holding one or more actuators 910 (for purposes of illustration and clarity, an actuator 910 is only shown in the second actuator chamber 908b in FIG. 9A). The actuator (not shown) in the first actuator chamber 908a can be configured to selectively control the flow of fluid through the first port 911, and/or to selectively control the flow of fluid through another port or aperture upstream from the first channel 922 (e.g., at one or more intermediate ports or apertures positioned fluidly between the first port 911 and the first channel 922). The actuator 910 in the second actuator chamber 908b can be configured to selectively control the flow of fluid through the third port 917, and/or to selectively control the flow of fluid through another port or aperture upstream from the bypass channel 915 (e.g., at one or more intermediate ports or apertures positioned fluidly between the third port 917 and the bypass channel 915). In the illustrated embodiment, the second port 913 that provides access to the second channel 924 does not include a corresponding actuator for selectively controlling the flow of fluid therethrough, and therefore remains open/unblocked. The actuators 910 can be similar to or the same as the actuators 810 described with reference to FIGS. 8A-8D. For example, the actuators 910 can be shape memory actuators, such as those described with reference to FIGS. 5A, 5B, 7A, and 7B, and/or those described in U.S. Patent Application Publication Nos. US 2020/0229982 and US 2021/0251806, the disclosures of which were previously incorporated by reference herein.

FIG. 9B is a schematic diagram of the fluid resistor network 920 further demonstrating the relative fluid resistance of the various flow pathways through the fluid resistor network 920. As illustrated, the first channel 922, the first portion 924a of the second channel 924, and the second portion 924b of the second channel 924 are the primary sources of resistance through the network 920. That is, the ports 911, 913, and 917, the bypass channel 915, and the third channel 926 do not impart a meaningful resistance through the network 920. As a result, the network 920 provides three fluid flow paths through the system: a first flow path D between the first port 911 and the third channel 926 via the first channel 922, a second flow path E between the second port 913 and the third channel 926 via the first portion 924a and the second portion 924b of the second channel 924, and a third flow path F between the third port 917 and the third channel 926 via the bypass channel 925 and the second portion 924b of the second channel 924. In the illustrated embodiment, the first flow path D has the lowest resistance, the second flow path E has the highest resistance, and the third flow path F has an intermediate resistance between the first resistance and the second resistance. As one skilled in the art will appreciate from the disclosure herein, the channels can be arranged to provide different relative resistances than those depicted herein, and therefore the present technology is not limited to any particular configuration of channels, unless expressly noted otherwise. Indeed, additional examples of fluid resistor networks are described in Section C below.

Similar to the system 800 described above previously, the system 900 can be composed of multiple layers of material stacked and adhered together. For example, FIG. 9C is an exploded isometric view of the system 900 showing a first layer 902, a second layer 903, and a third layer 904. The first layer 902 includes the first port 911, the second port 913, and the third port 917, which can each be through holes extending between a first (e.g., upper) surface 951 and a second (e.g., lower) surface 952 of the first layer 902. Unlike the system 800, the majority of the fluid resistor network 220 (FIG. 9B) is defined within the first layer 902. That is, the first channel 922, the second channel 924, and the third channel 926 (not visible in FIG. 9C) are at least partially defined within the thickness of the first layer 902. For example, the first layer 902 defines the void space of the channels 922, 924, and 926. In such embodiments, the second layer 903 and/or the third layer 904 may provide at least a portion of the wall to the void space of the channels 922, 924, and 926 defined within the first layer 902.

As set forth above, the system 900 includes certain features expected to improve the manufacturability of the multi-layered system 900. For example, the second surface 952 of the first layer 902 can have a first layer step 953 dividing the first layer 902 into a first region 954 having a first thickness and a second region 955 having a second thickness that is greater than the first thickness. For example, the first region 954 may have a thickness that is between about 1/8 and about 3/4 of the thickness of the second region 955. The first surface 961 of the second layer 903 can also have a second layer step 963 dividing the second layer 903 into a first region 964 having a first thickness and a second region 965 having a second thickness that is less than the first thickness. For example, the second region 965 may have a thickness that is between about 1/8 and about 3/4 of the thickness of the first region 964. The first region 964 of the second layer can include the actuator chambers 908. The first regions 954, 964 have a combined thickness that is equal or at least approximately equal to the combined thickness of the second regions 955, 965.

When the first layer 902 and the second layer 903 are adhered together, the first region 954 of the first layer 902 aligns with (and is adhered to) the first region 964 of the second layer 903. Similarly, the second region 955 of the first layer 902 aligns with (and is adhered to) the second region 965 of the second layer 903. As a result, the first layer step 953 sits against the second layer step 963 when the first layer 902 is adhered to the second layer 903. Without being bound by theory, including layers of variable thickness is expected to improve the manufacturability of the system 900 because (1) the fluid resistor network 920 can exist in the same or substantially the same plane as the actuators 910, while (2) permitting the fluid resistor network 920 to be manufactured in a separate layer than the actuator housing 908. This is expected to be advantageous because the material properties best suited for forming the fluid resistor network 920 may be different than the material properties best suited for forming the actuator housing 908. For example, in some embodiments it is expected that the first layer 902 (and thus the fluid resistor network 920) is composed of a relatively flexible material such as silicone, whereas the second layer 903 (and thus the actuator housing 908) is composed of a relatively inflexible material (such as superelastic Nitinol, stainless steel, or other medical grade, stiff materials). This is also expected to be advantageous because it may result in having to adhere fewer layers of material together to form the system 900. In some embodiments, however, the fluid resistor network 920 is manufactured in multiple layers of relatively flexible material, as described above with reference to FIGS. 8A-8D, in addition to having the layered steps as described with reference to FIG. 9C.

### C. Additional Embodiments of Fluid Resistor Networks

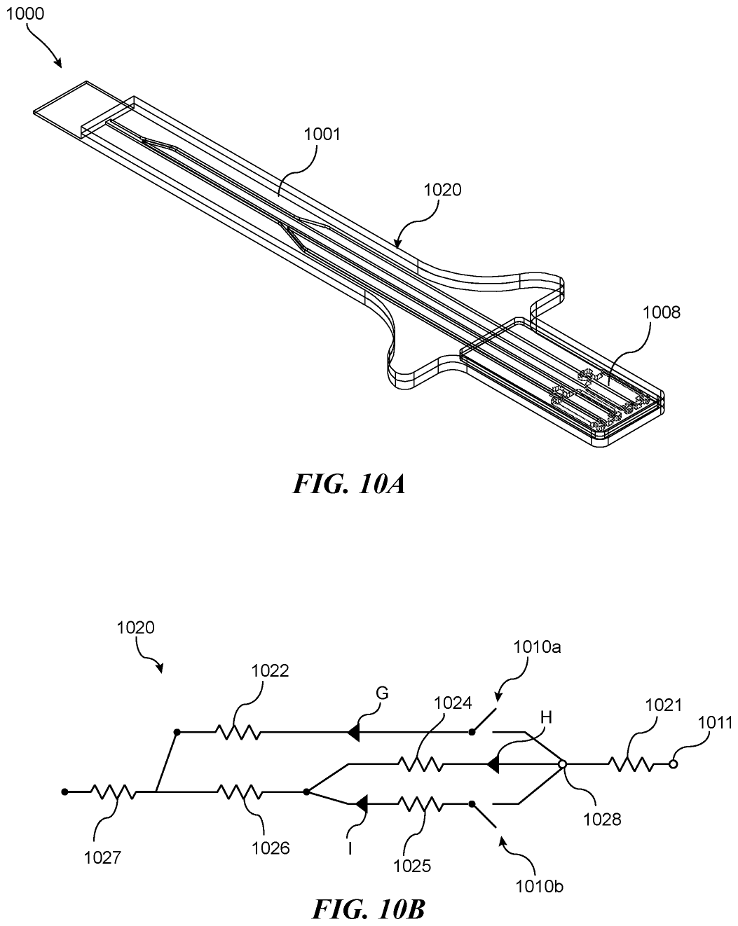

As set forth above, the present technology is not limited to the fluid resistor networks described and illustrated with respect to FIGS. 8A-9C. Indeed, as one skilled in the art will appreciate from the disclosure herein, numerous different flow pathways can be created through different layers of the shunting systems to provide different titratable therapy levels. FIGS. 10A and 10B, for example, illustrate another shunting system 1000 (“the system 1000”) configured in accordance with select embodiments of the present technology and having a different fluid resistor network than the systems 800 and 900 described with reference to FIGS. 8A-9C. More specifically, FIG. 10A is a perspective view of the system 1000 and FIG. 10B is a schematic representation of the fluidic resistor network shown in FIG. 10A. Similar to the systems 100 and 200 described previously, the system 1000 can be an adjustable shunt configured to drain fluid from a first body region to a second body region, such as to drain aqueous from an anterior chamber of a patient's eye.

Certain features of the system 1000 can be generally similar to corresponding features of the systems 800 and 900 described previously. For example, the system 1000 includes an elongated housing or shunting element 1001 having a network of fluid resistors 1020 (also referred to herein as “the fluid resistor network 1020”). Similar to the fluid resistor networks 820 and 920 described above, the fluid resistor network 1020 can be composed of a plurality of channels or lumens extending through shunting element 1001 to provide a plurality of flowpaths for draining fluid therethrough. However, as described in detail below with reference to FIG. 10B, the fluid resistor network 1020 of the system 1000 is different than the fluid resistor networks 820 and 920 described previously. The system 1000 can further include one or more actuator chambers 1008 configured to house corresponding actuators (not shown) for selectively controlling flow of fluid through the fluid resistor network 1020.

Referring now to FIG. 10B, the fluid resistor network 1020 includes a plurality of distinct channel segments: a first channel segment 1021, a second channel segment 1022, a third channel segment 1024, a fourth channel segment 1025, a fifth channel segment 1026, and a sixth channel segment 1027. The channel segments collectively define three different flow paths G-I through the fluid resistor network 1020, with each flow path having a different total resistance. Each of the three flow paths includes the first channel segment 1021 and the sixth channel segment 1027. That is, the first channel segment 1021 is a common inflow channel that collects fluid from a corresponding inlet port 1011, and the sixth channel segment 1027 is a common outflow channel that collects fluid from the second channel segment 1022 and the fifth channel segment 1026. In the illustrated embodiment, the first channel segment 1021 and the sixth channel segment 1027 impart a meaningful resistance to the fluid pathways. However, the resistance imparted by the first channel segment 1021 and the sixth channel 1027 is the same for each flow path. In other embodiments, the first channel segment 1021 and/or the sixth channel segment 1027 do not impart a meaningful resistance, relative to the total resistance of the flow pathways.

The fluid resistor network 1020 includes three parallel resistors: the second channel segment 1022, the third channel segment 1024, and the fourth channel segment 1025. Each of the parallel resistors defines a different flow path: a first flow path G through the second channel segment 1022, a second flow path H through the third channel segment 1024, and a third flow path I through the fourth channel segment 1025. One or more of the parallel resistors can be selectively gated by corresponding actuators. For example, in the illustrated embodiment the second channel segment 1022 is gated by a first actuator 1010a and the fourth channel segment 1025 is gated by a second actuator 1010b. The first and second actuators 1010a, 1010b can be non-invasively actuated to selectively open or close the second channel segment 1022 and the fourth channel segment 1025, respectively. In the illustrated embodiment, the third channel segment 1024 remains open. The third channel segment 1024 and the fourth channel segment 1025 can merge into the fifth channel segment 1026. The second channel segment 1022 and the fifth channel segment can merge into the sixth channel segment 1027, which serves as the common outflow channel for each flow path G-I, as described above. The resistances of each of the channel segments can be selected such that each flow path through the system 1000 has desired resistive properties. Moreover, as set forth above in Section A, different channel segments can be defined within different layers of the system 1000 to minimize the size of the system 1000 and/or improve the manufacturability of system 1000.

FIG. 11 is a cross-sectional view of a portion of a fluid resistor network 1100 configured in accordance with select embodiments of the present technology, and that can be implemented with any of the shunting systems described herein. More specifically, FIG. 11 is a cross-sectional view taken along an axial length of the fluid resistor network 1100. As illustrated, the fluid resistor network 1100 includes a first channel 1122 having a first (e.g., upper) wall 1122a and a second (e.g., lower) wall 1122b, and a second channel 1124 having a third (e.g., upper) wall 1124a and the fourth (e.g., lower) wall 1124b. Although only a cross-sectional view is illustrated in FIG. 11, the first channel 1122 and the second channel 1124 can have a shape generally similar to any of the channels described herein (e.g., linear, spiral, serpentine, etc.).

As illustrated, the void space of both the first channel 1122 and the second channel 1124 are substantially defined within a first layer 1102. However, the first layer 1102 does not fully enclose either the first channel 122 or the second channel 1124. Rather, the first channel 1122 is enclosed when the first layer 1102 is sealingly coupled to a second layer 1103 (e.g., the second layer 1103 includes the first wall 1122a of the first channel 1122), and the second channel 1124 is fully enclosed when the first layer 1102 is sealingly coupled to a third layer 1104 (e.g, the third layer 1104 includes the fourth wall 1124b). However, despite being defined within the same layer, the first channel 1122 and the second channel 1124 are not coplanar within the first layer 1102. More specifically, a “top” of the first channel 1122 (e.g., the first wall 1122a), a “bottom” of the first channel 1122 (e.g., the second wall 1122b), a “top” of the second channel 1124 (e.g., the third wall 1124a), and a “bottom” of the second channel 1124 (e.g., the fourth wall 1124b) each occupy different planes (labeled as planes A-D, respectively) that extend parallel to an axial length of the system (e.g., planes A-D extend into the page along the dashed line in the view shown in FIG. 11). Without being bound by theory, such a configuration may be easier to manufacture than configurations that require one or more channels be fully formed within a single layer, while still minimizing the overall height of the system.

The present technology can include other embodiments in which channels, or portions of channels, occupy different planes within one or more layers. For example, some embodiments include a shunt body having at least a first channel extending at least partially through the shunt body and a second channel extending at least partially through the shunt body. The first channel can have a first (e.g., upper) wall and a second (e.g., lower) wall, and the second channel can have a third (e.g., upper) wall and a fourth (e.g., lower) wall, as described above with reference to FIG. 11. Each of the first wall, the second wall, the third wall, and the fourth wall can extend in different planes, with each plane being parallel to an axial length of the shunt body. That is, the various walls can exist at different “heights” in the shunt body. In some embodiments, this can be accomplished using the configuration shown in FIG. 11, in which the first channel 1122 and the second 1124 are defined within the first layer 1102. In other embodiments, however, the first channel and the second channel may be defined or at least partially defined within different layers, and/or within two or more layers, such as in the embodiment described above with reference to FIGS. 8A-8D.

The fluid resistor networks 1000 and 1100 are provided merely as additional examples of variations of the fluid resistor networks described herein. As one skilled in the art will appreciate, the present technology can include other fluid resistor networks beyond those expressly described and illustrated herein. Indeed, additional examples of fluid resistor networks that can be implemented with the present technology are described in U.S. Patent Application Publication No. US 2022/0142818, the disclosure of which is incorporated by reference herein.

The present technology may provide additional advantages beyond those explicitly described herein. For example, the present technology may provide enhanced surface quality for the drainage assemblies, actuation assemblies, and/or shunting systems; better mechanical properties of the drainage assemblies, actuation assemblies, and/or shunting systems; and/or enable a larger selection of materials to be used for fabricating the drainage assemblies, actuation assemblies, and/or shunting systems.

### Examples

Several aspects of the present technology are set forth in the following examples:
1. An implantable shunt for treating a patient, the implantable shunt comprising:
a first layer;
a second layer sealingly coupled to the first layer; and
a network of fluid resistors including at least a first channel having a first resistance and a second channel in parallel with the first channel and having a second resistance,
wherein-
   the first channel is at least partially defined within the first layer,
   the second channel is at least partially defined within the second layer, and
   when the implantable shunt is implanted in the patient, the first channel and the second channel are each configured to drain fluid from a first body region toward a second body region in the patient.
2. The implantable shunt of example 1 wherein:
the first layer defines a first plane,
the second layer defines a second plane, and
the first plane and the second plane are parallel.
3. The implantable shunt of example 2 wherein the first plane and the second plane are parallel to a longitudinal axis of the implantable shunt.
4. The implantable shunt of example 2 wherein the first plane and the second plane are perpendicular to a longitudinal axis of the implantable shunt.
5. The implantable shunt of any of examples 1-4 wherein the first channel is fluidically in parallel with the second channel such that the first channel defines a first flow path at least partially through the shunt and the second channel defines a second flow path at least partially through the shunt that is distinct from the first flow path.
6. The implantable shunt of any of examples 1-5 wherein the first resistance is different than the second resistance.
7. The implantable shunt of any of examples 1-6 wherein the second channel is longer than the first channel, and wherein the second resistance is greater than the first resistance.
8. The implantable shunt of any of examples 1-7 wherein at least one of the first channel or the second channel is spiral and/or serpentine shaped.
9. The implantable shunt of example 8 wherein both the first channel and the second channel are spiral and/or serpentine shaped.
10. The implantable shunt of any of examples 1-9 wherein the implantable shunt is an intraocular shunt, and wherein the first body region is an anterior chamber of an eye of the patient.
11. An implantable shunt for treating a patient, the implantable shunt comprising:
a first layer;
a second layer sealingly coupled to the first layer; and
a network of fluid resistors including at least a first channel having a first resistance and a second channel in parallel with the first channel and having a second resistance,
wherein-
   the first channel is at least partially defined within the first layer,
   the second channel includes a first portion at least partially defined within the first layer and a second portion at least partially defined within the second layer, and
   when the implantable shunt is implanted in the patient, the first channel and the second channel are each configured to drain fluid from a first body region toward a second body region in the patient.
12. The implantable shunt of example 11 wherein the first channel is fluidically in parallel with the second channel such that the first channel defines a first flow path at least partially through the shunt and the second channel defines a second flow path at least partially through the shunt that is distinct from the first flow path.
13. The implantable shunt of example 11 or 12 wherein the first resistance is different than the second resistance.
14. The implantable shunt of any of examples 11-13 wherein the second channel is longer than the first channel, and wherein the second resistance is greater than the first resistance.
15. The implantable shunt of any of examples 11-14 wherein the first channel is substantially straight, and the second channel is spiral and/or serpentine shaped.
16. The implantable shunt of any of examples 11-15 wherein the network of fluid resistors further includes a third channel.
17. The implantable shunt of example 16 wherein the third channel is configured to receive fluid from both the first channel and the second channel.
18. The implantable shunt of example 16 or example 17 wherein the third channel has a third resistance, the third resistance being less than the first resistance and the second resistance.
19. The implantable shunt of any of examples 16-18 wherein the third channel is at least partially defined within the first layer.
20. The implantable shunt of any of examples 16-18 wherein the third channel is at least partially defined within the second layer.
21. The implantable shunt of any of examples 11-20 wherein the fluid resistor network further includes a bypass channel, the bypass channel extending between an inflow port and a portion of the second channel between the first portion and the second portion.
22. The implantable shunt of any of examples 11-21 wherein:
the first layer defines a first plane,
the second layer defines a second plane, and
the first plane and the second plane are parallel to a longitudinal axis of the implantable shunt.
23. The implantable shunt of any of examples 11-22 wherein the first layer and the second layer each have a thickness of less than about 100 microns.
24. The implantable shunt of any of examples 11-22 wherein the first layer and the second layer each have a thickness of less than about 50 microns.
25. The implantable shunt of any of examples 11-22 wherein the first layer and the second layer each have a thickness of less than about 25 microns.
26. The implantable shunt of any of examples 11-25, further comprising a third layer.
27. The implantable shunt of example 26 wherein the third layer is composed of a different material than the first layer and/or the second layer.
28. The implantable shunt of example 26 or 27 wherein the third layer includes an actuator housing configured to house an actuator operable to selectively control the flow of fluid through the first channel and/or the second channel.
29. The implantable shunt of example 26 or 27 wherein the third layer is positioned between the first layer and the second layer, and wherein the third layer includes a connector fluidly coupling the first portion of the second channel and the second portion of the second channel.
30. The implantable shunt of any of examples 11-29 wherein the implantable shunt is an intraocular shunt, and wherein the first body region is an anterior chamber of an eye of the patient.
31. An implantable shunt for treating a patient, the implantable shunt comprising:
a first flat layer having a first thickness of between 10 microns and 500 microns;
a second flat layer having a second thickness of between 10 microns and 500 microns, wherein the second flat layer is sealingly coupled to the first flat layer; and
a network of fluid resistors including at least a first channel having a first resistance and a second channel in parallel with the first channel and having a second resistance;
wherein-
   the first channel is at least partially defined within the first layer,
   the second channel is at least partially defined within the second layer, and
   the first channel and the second channel are each configured to drain fluid from a first body region toward a second body region in the patient when the implantable shunt is implanted in the patient.
32. The implantable shunt of example 31 wherein the first channel is fluidically in parallel with the second channel such that the first channel defines a first flow path at least partially through the shunt and the second channel defines a second flow path at least partially through the shunt that is distinct from the first flow path.
33. The implantable shunt of example 31 or 32 wherein the first resistance is different than the second resistance.
34. The implantable shunt of any of examples 31-33 wherein:
the first layer defines a first plane,
the second layer defines a second plane, and
the first plane and the second plane are parallel to a longitudinal axis of the implantable shunt.
35. The implantable shunt of any of examples 31-34 wherein the first thickness is between 10 microns and 100 microns, and wherein the second thickness is between 10 microns and 100 microns.
36. The implantable shunt of any of examples 31-34 wherein the first thickness is between 10 microns and 50 microns, and wherein the second thickness is between 10 microns and 50 microns.
37. The implantable shunt of any of examples 31-36 wherein the first thickness and the second thickness are about the same.
38. The implantable shunt of any of examples 31-36 wherein the first thickness and the second thickness are different.
39. The implantable shunt of any of examples 31-38, further comprising a third layer.
40. The implantable shunt of example 39 wherein the third layer is composed of a different material than the first layer and/or the second layer.
41. The implantable shunt of example 39 or 40 wherein the third layer includes an actuator housing configured to house an actuator operable to selectively control the flow of fluid through the first channel and/or the second channel.
42. The implantable shunt of any of examples 31-41 wherein the implantable shunt is an intraocular shunt, and wherein the first body region is an anterior chamber of an eye of the patient.
43. An implantable shunt for treating a patient, the implantable shunt comprising:
a shunt body;
a first channel extending at least partially through the shunt body, the first channel having at least a first wall and a second wall that at least partially define a void space of the first channel; and
a second channel extending at least partially through the shunt body, the second channel having at least a third wall and a fourth wall that at least partially define a void space of the second channel,
wherein each of the first wall, the second wall, the third wall, and the fourth wall extend in different planes parallel to an axial length of the shunt body.
44. The implantable shunt of example 43 wherein the shunt body comprises a plurality of sealingly coupled layers.
45. The implantable shunt of example 44 wherein the void space of the first channel and the void space of the second channel are defined within separate layers.
46. The implantable shunt of example 44 wherein the void space of the first channel and the void space of the second channel are defined with the same layer.
47. The implantable shunt of example 43 wherein the shunt body includes:
a first layer, wherein the first layer at least partially defines the void space of the first channel; and
a second layer, wherein the second layer includes at least one of the first wall or the second wall.
48. The implantable shunt of example 43 wherein the shunt body includes:
a first layer at least partially defining the void space of the first channel and the void space of the second channel;
a second layer sealing coupled to a first side of the first layer, wherein the second layer includes the first wall or the second wall; and
a third layer sealing coupled to a second side of the first layer, wherein the second layer includes the third wall or the fourth wall.
49. A system for shunting fluid, the system comprising:
a carriage element including a slot and an at least partially hollow interior; and
a plurality of drainage elements positioned within the interior of the carriage element, wherein each drainage element of the plurality of drainage elements includes:
   an inlet,
   a drainage lumen portion, and
   a channel fluidly coupling the inlet and the drainage lumen portion;
wherein individual drainage lumen portions of individual drainage elements at least partially align to form a common drainage lumen extending through at least a portion of the carriage element.
50. The system of example 49 wherein the plurality of drainage elements are linearly aligned in the carriage in a stacked configuration.
51. The system of example 49 or 50 wherein the channel has a serpentine shape, a spiral shape, a zig-zag shape, a curvilinear shape, or a rectilinear shape.
52. The system of any of examples 49-51 wherein the drainage lumen has a rectangular shape, a circular shape, an oval shape, a curvilinear shape, triangular shape, a square shape, a rectangular shape, a rectilinear shape, a pentagonal shape, or a hexagonal shape.
53. The system of any of examples 49-52 wherein the carriage element has a circular shape, an oval shape, a triangular shape, a square shape, a rectangular shape, a pentagonal shape, a hexagonal shape, a curvilinear shape, or a rectilinear shape, and wherein individual ones of the one or more drainage elements have a shape that corresponds to the carriage element shape.
54. The system of any of examples 49-53 wherein each drainage element further includes:
an interface surface, and
a projection extending from the interface surface,
wherein the projection includes the inlet, and
wherein an opening of the inlet is perpendicular to the interface surface.
55. The system of any of examples 49-54 wherein the one or more drainage elements include a first drainage element having a first drainage lumen portion and a second drainage element having a second drainage lumen portion, and wherein the first and second drainage lumen portions at least partially form the common drainage lumen.
56. The system of any of examples 49-55 wherein the one or more drainage elements are coupled to each other via at least one of the following: an adhesive, ultrasonic welding thermal fusion, thermal reflow, and/or an inherent adhesive property of one or more of the drainage elements.
57. The system of any of examples 49-56 wherein the one or more drainage elements include a first drainage element and a second drainage element, and wherein:
the first drainage element includes a first channel having a first length corresponding to a first fluid resistance; and
the second drainage element includes a second channel having a second length corresponding to a second fluid resistance.
58. The system of example 57 wherein the first length is greater than the second length, and wherein the first length corresponds to a first fluid resistance greater than the second fluid resistance.
59. The system of any of examples 49-58 wherein the individual ones of the one or more drainage elements further include:
one or more alignment elements, and
one or more alignment apertures configured to receive a corresponding one of the one or more alignment elements, wherein the one or more alignment elements and the one or more alignment apertures are configured to linearly align the plurality of drainage elements in a stacked configuration.
60. The system of any of examples 49-59, further comprising an actuation assembly having a plurality of actuators, wherein each actuator of the plurality of actuators is configured to selectively control the flow of fluid through a corresponding inlet.
61. The system of any of examples 49-60 wherein the channel has a length greater than a height of the corresponding drainage element.
62. An actuation assembly for use with a shunting system, the actuation assembly comprising:
a housing including one or more wells; and
for individual ones of the one or more wells:
   an actuator positioned within the well, the actuator including:
      a gating element having a central portion, a first end portion extending from a first side of the central portion and generally perpendicular to a longitudinal axis of the central portion, and a second end portion extending form a second side of the central portion and generally perpendicular to the longitudinal axis of the central portion,
      one or more first actuation elements extending between the first end portion and the housing, and
      one or more second actuation elements extending between the second end portion and the housing,
      wherein the plurality of first actuation elements are configured to slidably move the gating element in a first direction and the plurality of second actuation elements are configured to slidably move the gating element in a second direction.
63. The actuation assembly of example 62 wherein the one or more first and second actuation elements are composed of nitinol.
64. The actuation assembly of example 62 or example 63, further comprising a priming element coupled to the one or more second actuation elements and opposite the second end portion.
65. The actuation assembly of example 64 wherein individual ones of the one or more wells further include a priming surface, and wherein, when the priming element is coupled to the priming surface, the one or more second actuation elements are configured to be deformed relative to a preferred geometry.
66. The actuation assembly of any of examples 62-65 wherein at least one of the one or more first actuation elements and/or the one or more second actuation elements has an hourglass shape.
67. The actuation assembly of any of examples 62-66 wherein respective surfaces of the gating element, the plurality of first actuation elements, and the plurality of second actuation elements are coplanar.

### Conclusion

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the intraocular shunts described herein may be combined with any of the features of the other intraocular shunts described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. An implantable shunt for treating a patient, the implantable shunt comprising:
a first layer;
a second layer sealingly coupled to the first layer; and
a network of fluid resistors including at least a first channel having a first resistance and a second channel in parallel with the first channel and having a second resistance,
wherein-
the first channel is at least partially defined within the first layer,
the second channel is at least partially defined within the second layer, and
when the implantable shunt is implanted in the patient, the first channel and the second channel are each configured to drain fluid from a first body region toward a second body region in the patient.

2. The implantable shunt of claim 1 wherein:
the first layer defines a first plane,
the second layer defines a second plane, and
the first plane and the second plane are parallel.

3. The implantable shunt of claim 2 wherein the first plane and the second plane are parallel to a longitudinal axis of the implantable shunt.

4. The implantable shunt of claim 2 wherein the first plane and the second plane are perpendicular to a longitudinal axis of the implantable shunt.

5. The implantable shunt claim 1 wherein the first channel is fluidically in parallel with the second channel such that the first channel defines a first flow path at least partially through the shunt and the second channel defines a second flow path at least partially through the shunt that is distinct from the first flow path.

6. The implantable shunt of claim 1 wherein the first resistance is different than the second resistance.

7. The implantable shunt of claim 1 wherein the second channel is longer than the first channel, and wherein the second resistance is greater than the first resistance.

8. The implantable shunt of claim 1 wherein at least one of the first channel or the second channel is spiral and/or serpentine shaped.

9. The implantable shunt of claim 8 wherein both the first channel and the second channel are spiral and/or serpentine shaped.

10. The implantable shunt of claim 1 wherein the implantable shunt is an intraocular shunt, and wherein the first body region is an anterior chamber of an eye of the patient.

11. An implantable shunt for treating a patient, the implantable shunt comprising:
a first layer;
a second layer sealingly coupled to the first layer; and
a network of fluid resistors including at least a first channel having a first resistance and a second channel in parallel with the first channel and having a second resistance,
wherein-
the first channel is at least partially defined within the first layer,
the second channel includes a first portion at least partially defined within the first layer and a second portion at least partially defined within the second layer, and
when the implantable shunt is implanted in the patient, the first channel and the second channel are each configured to drain fluid from a first body region toward a second body region in the patient.

12. The implantable shunt of claim 11 wherein the first channel is fluidically in parallel with the second channel such that the first channel defines a first flow path at least partially through the shunt and the second channel defines a second flow path at least partially through the shunt that is distinct from the first flow path.

13. The implantable shunt of claim 11 wherein the first resistance is different than the second resistance.

14. The implantable shunt of claim 11 wherein the second channel is longer than the first channel, and wherein the second resistance is greater than the first resistance.

15. The implantable shunt of claim 11 wherein the first channel is substantially straight, and the second channel is spiral and/or serpentine shaped.

16. The implantable shunt of claim 11 wherein the network of fluid resistors further includes a third channel.

17. The implantable shunt of claim 16 wherein the third channel is configured to receive fluid from both the first channel and the second channel.

18. The implantable shunt of claim 16 wherein the third channel has a third resistance, the third resistance being less than the first resistance and the second resistance.

19. The implantable shunt of claim 16 wherein the third channel is at least partially defined within the first layer.

20. The implantable shunt of claim 16 wherein the third channel is at least partially defined within the second layer.

21. The implantable shunt of claim 11 wherein the fluid resistor network further includes a bypass channel, the bypass channel extending between an inflow port and a portion of the second channel between the first portion and the second portion.

22. The implantable shunt of claim 11 wherein:
the first layer defines a first plane,
the second layer defines a second plane, and
the first plane and the second plane are parallel to a longitudinal axis of the implantable shunt.

23. The implantable shunt of claim 11 wherein the first layer and the second layer each have a thickness of less than about 100 microns.

24. The implantable shunt of claim 11 wherein the first layer and the second layer each have a thickness of less than about 50 microns.

25. The implantable shunt of claim 11 wherein the first layer and the second layer each have a thickness of less than about 25 microns.

26. The implantable shunt of claim 11, further comprising a third layer.

27. The implantable shunt of claim 26 wherein the third layer is composed of a different material than the first layer and/or the second layer.

28. The implantable shunt of claim 26 wherein the third layer includes an actuator housing configured to house an actuator operable to selectively control the flow of fluid through the first channel and/or the second channel.

29. The implantable shunt of claim 26 wherein the third layer is positioned between the first layer and the second layer, and wherein the third layer includes a connector fluidly coupling the first portion of the second channel and the second portion of the second channel.

30. The implantable shunt of claim 11 wherein the implantable shunt is an intraocular shunt, and wherein the first body region is an anterior chamber of an eye of the patient.

31. An implantable shunt for treating a patient, the implantable shunt comprising:
a first flat layer having a first thickness of between 10 microns and 500 microns;
a second flat layer having a second thickness of between 10 microns and 500 microns, wherein the second flat layer is sealingly coupled to the first flat layer; and
a network of fluid resistors including at least a first channel having a first resistance and a second channel in parallel with the first channel and having a second resistance;
wherein-
the first channel is at least partially defined within the first layer,
the second channel is at least partially defined within the second layer, and
the first channel and the second channel are each configured to drain fluid from a first body region toward a second body region in the patient when the implantable shunt is implanted in the patient.

32. The implantable shunt of claim 31 wherein the first channel is fluidically in parallel with the second channel such that the first channel defines a first flow path at least partially through the shunt and the second channel defines a second flow path at least partially through the shunt that is distinct from the first flow path.

33. The implantable shunt of claim 31 wherein the first resistance is different than the second resistance.

34. The implantable shunt of claim 31 wherein:
the first layer defines a first plane,
the second layer defines a second plane, and
the first plane and the second plane are parallel to a longitudinal axis of the implantable shunt.

35. The implantable shunt of claim 31 wherein the first thickness is between 10 microns and 100 microns, and wherein the second thickness is between 10 microns and 100 microns.

36. The implantable shunt of claim 31 wherein the first thickness is between 10 microns and 50 microns, and wherein the second thickness is between 10 microns and 50 microns.

37. The implantable shunt of claim 31 wherein the first thickness and the second thickness are about the same.

38. The implantable shunt of claim 31 wherein the first thickness and the second thickness are different.

39. The implantable shunt of claim 31, further comprising a third layer.

40. The implantable shunt of claim 39 wherein the third layer is composed of a different material than the first layer and/or the second layer.

41. The implantable shunt of claim 39 wherein the third layer includes an actuator housing configured to house an actuator operable to selectively control the flow of fluid through the first channel and/or the second channel.

42. The implantable shunt of claim 31 wherein the implantable shunt is an intraocular shunt, and wherein the first body region is an anterior chamber of an eye of the patient.

43. An implantable shunt for treating a patient, the implantable shunt comprising:
a shunt body;
a first channel extending at least partially through the shunt body, the first channel having at least a first wall and a second wall that at least partially define a void space of the first channel; and
a second channel extending at least partially through the shunt body, the second channel having at least a third wall and a fourth wall that at least partially define a void space of the second channel,
wherein each of the first wall, the second wall, the third wall, and the fourth wall extend in different planes parallel to an axial length of the shunt body.

44. The implantable shunt of claim 43 wherein the shunt body comprises a plurality of sealingly coupled layers.

45. The implantable shunt of claim 44 wherein the void space of the first channel and the void space of the second channel are defined within separate layers.

46. The implantable shunt of claim 44 wherein the void space of the first channel and the void space of the second channel are defined with the same layer.

47. The implantable shunt of claim 43 wherein the shunt body includes:
a first layer, wherein the first layer at least partially defines the void space of the first channel; and
a second layer, wherein the second layer includes at least one of the first wall or the second wall.

48. The implantable shunt of claim 43 wherein the shunt body includes:
a first layer at least partially defining the void space of the first channel and the void space of the second channel;
a second layer sealing coupled to a first side of the first layer, wherein the second layer includes the first wall or the second wall; and
a third layer sealing coupled to a second side of the first layer, wherein the second layer includes the third wall or the fourth wall.
